SEQUENCE LISTING (647 sequences)

SEQ ID NO: 1 - protein, 15 aa, Artificial Sequence
  Description of Artificial Sequence Synthetic peptide
LDSEISMYTFSRMKY

SEQ ID NO: 2 - protein, 15 aa, Artificial Sequence
  Description of Artificial Sequence Synthetic peptide
ISMYTFSRMKYNICM

SEQ ID NO: 3 - protein, 15 aa, Artificial Sequence
  Description of Artificial Sequence Synthetic peptide
TMNEYSVPRTLQARF

SEQ ID NO: 4 - protein, 15 aa, Artificial Sequence
  Description of Artificial Sequence Synthetic peptide
KGVNNPYGLYSRMCR

SEQ ID NO: 5 - protein, 15 aa, Artificial Sequence
  Description of Artificial Sequence Synthetic peptide
IPTYGTPDWDEWWSQ

SEQ ID NO: 6 - protein, 15 aa, Artificial Sequence
  Description of Artificial Sequence Synthetic peptide
MSCWGNLPLMRRQYL

SEQ ID NO: 7 - protein, 15 aa, Artificial Sequence
  Description of Artificial Sequence Synthetic peptide
NPYGLYSRMCRQPFN

SEQ ID NO: 8 - protein, 15 aa, Artificial Sequence
  Description of Artificial Sequence Synthetic peptide
LAHYLDFAKPFPCQK

SEQ ID NO: 9 - protein, 15 aa, Artificial Sequence
  Description of Artificial Sequence Synthetic peptide
MPEMYNNLCKPPYKL

SEQ ID NO: 10 - protein, 15 aa, Artificial Sequence
  Description of Artificial Sequence Synthetic peptide
LGLERSAWGNIPLMR

SEQ ID NO: 11 - protein, 15 aa, Artificial Sequence
  Description of Artificial Sequence Synthetic peptide
MVYNIPKKRYWLFKG

SEQ ID NO: 12 - protein, 15 aa, Artificial Sequence
  Description of Artificial Sequence Synthetic peptide
RSAWGNIPLMRKAYL

SEQ ID NO: 13 - protein, 15 aa, Artificial Sequence
  Description of Artificial Sequence Synthetic peptide
HNQPYHICRGFTCFK

SEQ ID NO: 14 - protein, 15 aa, Artificial Sequence
  Description of Artificial Sequence Synthetic peptide
PTYGTDEWEQWWNAF

SEQ ID NO: 15 - protein, 15 aa, Artificial Sequence
  Description of Artificial Sequence Synthetic peptide
GTPDWDYWWSQFNSY

SEQ ID NO: 16 - protein, 15 aa, Artificial Sequence
  Description of Artificial Sequence Synthetic peptide
LIWCRPVSDFHPCIQ

SEQ ID NO: 17 - protein, 15 aa, Artificial Sequence
  Description of Artificial Sequence Synthetic peptide
LGLDMTCWGNLPLMR

SEQ ID NO: 18 - protein, 15 aa, Artificial Sequence
  Description of Artificial Sequence Synthetic peptide
TMNEYLVPATLAPRF

SEQ ID NO: 19 - protein, 15 aa, Artificial Sequence
  Description of Artificial Sequence Synthetic peptide
YLVPATLAPRFHKTV

SEQ ID NO: 20 - protein, 15 aa, Artificial Sequence
  Description of Artificial Sequence Synthetic peptide
IPTYGTPDWDYWWSQ

SEQ ID NO: 21 - protein, 15 aa, Artificial Sequence
  Description of Artificial Sequence Synthetic peptide
MTCWGNLPLMRTKYL

SEQ ID NO: 22 - protein, 15 aa, Artificial Sequence
  Description of Artificial Sequence Synthetic peptide
IDCYCFDCFRQWFGC

SEQ ID NO: 23 - protein, 15 aa, Artificial Sequence
  Description of Artificial Sequence Synthetic peptide
KPPVWIECYCYKCYR

SEQ ID NO: 24 - protein, 15 aa, Artificial Sequence
  Description of Artificial Sequence Synthetic peptide
QSSQVYCKDLCCNKF

SEQ ID NO: 25 - protein, 15 aa, Artificial Sequence
  Description of Artificial Sequence Synthetic peptide
HCILSKYHKEKYKIY

SEQ ID NO: 26 - protein, 15 aa, Artificial Sequence
  Description of Artificial Sequence Synthetic peptide
CIHGYNHECQCIHCI

SEQ ID NO: 27 - protein, 15 aa, Artificial Sequence
  Description of Artificial Sequence Synthetic peptide
KQKNCLTWGECFCYQ

SEQ ID NO: 28 - protein, 15 aa, Artificial Sequence
  Description of Artificial Sequence Synthetic peptide
DYMQSGYNARFCRGP

SEQ ID NO: 29 - protein, 15 aa, Artificial Sequence
  Description of Artificial Sequence Synthetic peptide
SGYNARFCRGPGCML

SEQ ID NO: 30 - protein, 15 aa, Artificial Sequence
  Description of Artificial Sequence Synthetic peptide
WIECYCYRCYREWFG

SEQ ID NO: 31 - protein, 15 aa, Artificial Sequence
  Description of Artificial Sequence Synthetic peptide
YCFLDKRHKQKYKIF

SEQ ID NO: 32 - protein, 15 aa, Artificial Sequence
  Description of Artificial Sequence Synthetic peptide
ELCCNFPPRKYRLVG

SEQ ID NO: 33 - protein, 15 aa, Artificial Sequence
  Description of Artificial Sequence Synthetic peptide
KPPMWIECYCYRCYR

SEQ ID NO: 34 - protein, 15 aa, Artificial Sequence
  Description of Artificial Sequence Synthetic peptide
FGTWNSSEVSCDFPP

SEQ ID NO: 35 - protein, 15 aa, Artificial Sequence
  Description of Artificial Sequence Synthetic peptide
PLCPDTLYCKDWPIC

SEQ ID NO: 36 - protein, 15 aa, Artificial Sequence
  Description of Artificial Sequence Synthetic peptide
IDCYCFDCFRQWFGL

SEQ ID NO: 37 - protein, 15 aa, Artificial Sequence
  Description of Artificial Sequence Synthetic peptide
EKKMLPCYSTARIPL

SEQ ID NO: 38 - protein, 15 aa, Artificial Sequence
  Description of Artificial Sequence Synthetic peptide
LPCYSTARIPLPNLY

SEQ ID NO: 39 - protein, 15 aa, Artificial Sequence
  Description of Artificial Sequence Synthetic peptide
AAGAANLFGPPVEKQ

SEQ ID NO: 40 - protein, 15 aa, Artificial Sequence
  Description of Artificial Sequence Synthetic peptide
TVDMMFRQFLQPQKP

SEQ ID NO: 41 - protein, 15 aa, Artificial Sequence
  Description of Artificial Sequence Synthetic peptide
ATTGNFQSRGLPYPM

SEQ ID NO: 42 - protein, 15 aa, Artificial Sequence
  Description of Artificial Sequence Synthetic peptide
DPDMMRYVDRYGQLQ

SEQ ID NO: 43 - protein, 15 aa, Artificial Sequence
  Description of Artificial Sequence Synthetic peptide
FNYRTMYPDGTIFPK

SEQ ID NO: 44 - protein, 15 aa, Artificial Sequence
  Description of Artificial Sequence Synthetic peptide
FNYRTTYPHGTIFPK

SEQ ID NO: 45 - protein, 15 aa, Artificial Sequence
  Description of Artificial Sequence Synthetic peptide
GMFTNRCGSQQWRGL

SEQ ID NO: 46 - protein, 15 aa, Artificial Sequence
  Description of Artificial Sequence Synthetic peptide
GMFTNRSGFQQWRGL

SEQ ID NO: 47 - protein, 15 aa, Artificial Sequence
  Description of Artificial Sequence Synthetic peptide
MRYVDRYGQLQTQML

SEQ ID NO: 48 - protein, 15 aa, Artificial Sequence
  Description of Artificial Sequence Synthetic peptide
PDMMRYVDRYGQSQT

SEQ ID NO: 49 - protein, 15 aa, Artificial Sequence
  Description of Artificial Sequence Synthetic peptide
PGDPDMMRYVDRYGQ

SEQ ID NO: 50 - protein, 15 aa, Artificial Sequence
  Description of Artificial Sequence Synthetic peptide
PNLNEDLTCGNIPMW

SEQ ID NO: 51 - protein, 15 aa, Artificial Sequence
  Description of Artificial Sequence Synthetic peptide
YLYKNKAYPVECWVP

SEQ ID NO: 52 - protein, 15 aa, Artificial Sequence
  Description of Artificial Sequence Synthetic peptide
LPGDPDMMRYVDRYG

SEQ ID NO: 53 - protein, 15 aa, Artificial Sequence
  Description of Artificial Sequence Synthetic peptide
GMFTNRSCSQQWRGL

SEQ ID NO: 54 - protein, 15 aa, Artificial Sequence
  Description of Artificial Sequence Synthetic peptide
AKLDKDGNYPIEVWC

SEQ ID NO: 55 - protein, 15 aa, Artificial Sequence
  Description of Artificial Sequence Synthetic peptide
PDMMRYVDKYGQLQT

SEQ ID NO: 56 - protein, 15 aa, Artificial Sequence
  Description of Artificial Sequence Synthetic peptide
WVADPSRNDNCRYFG

SEQ ID NO: 57 - protein, 15 aa, Artificial Sequence
  Description of Artificial Sequence Synthetic peptide
KAYLDKNNAYPVECW

SEQ ID NO: 58 - protein, 15 aa, Artificial Sequence
  Description of Artificial Sequence Synthetic peptide
PLEMQGVLMNYRTKY

SEQ ID NO: 59 - protein, 15 aa, Artificial Sequence
  Description of Artificial Sequence Synthetic peptide
AFVNNIHYLDPRHWG

SEQ ID NO: 60 - protein, 15 aa, Artificial Sequence
  Description of Artificial Sequence Synthetic peptide
YQLETGIPGIPDWLF

SEQ ID NO: 61 - protein, 15 aa, Artificial Sequence
  Description of Artificial Sequence Synthetic peptide
MAFSLDPLQWENSLL

SEQ ID NO: 62 - protein, 15 aa, Artificial Sequence
  Description of Artificial Sequence Synthetic peptide
MAVDLYRPDDYYDIL

SEQ ID NO: 63 - protein, 15 aa, Artificial Sequence
  Description of Artificial Sequence Synthetic peptide
YNLETGIPGVPDWVF

SEQ ID NO: 64 - protein, 15 aa, Artificial Sequence
  Description of Artificial Sequence Synthetic peptide
EHCTYHICKGFQCFK

SEQ ID NO: 65 - protein, 15 aa, Artificial Sequence
  Description of Artificial Sequence Synthetic peptide
FGTWNSSEVGCDFPP

SEQ ID NO: 66 - protein, 15 aa, Artificial Sequence
  Description of Artificial Sequence Synthetic peptide
FGTWNSSEVCADFPL

SEQ ID NO: 67 - protein, 15 aa, Artificial Sequence
  Description of Artificial Sequence Synthetic peptide
HCPCLMCMLKLRHKN

SEQ ID NO: 68 - protein, 15 aa, Artificial Sequence
  Description of Artificial Sequence Synthetic peptide
LMCMLKLRHRNRKFL

SEQ ID NO: 69 - protein, 15 aa, Artificial Sequence
  Description of Artificial Sequence Synthetic peptide
LMCMLKLRHKNRKFL

SEQ ID NO: 70 - protein, 15 aa, Artificial Sequence
  Description of Artificial Sequence Synthetic peptide
SPERKMLPCYSTARI

SEQ ID NO: 71 - protein, 15 aa, Artificial Sequence
  Description of Artificial Sequence Synthetic peptide
ALELQGVVCNYRTKY

SEQ ID NO: 72 - protein, 15 aa, Artificial Sequence
  Description of Artificial Sequence Synthetic peptide
YLDENKAYPVECWVP

SEQ ID NO: 73 - protein, 15 aa, Artificial Sequence
  Description of Artificial Sequence Synthetic peptide
YLDRNKAYPVECWVP

SEQ ID NO: 74 - protein, 15 aa, Artificial Sequence
  Description of Artificial Sequence Synthetic peptide
TTVLLDEFGGGPLCK

SEQ ID NO: 75 - protein, 15 aa, Artificial Sequence
  Description of Artificial Sequence Synthetic peptide
TTVLLDEYGVGPLCK

SEQ ID NO: 76 - protein, 15 aa, Artificial Sequence
  Description of Artificial Sequence Synthetic peptide
FGVGPLCKGANLYLS

SEQ ID NO: 77 - protein, 15 aa, Artificial Sequence
  Description of Artificial Sequence Synthetic peptide
GDPDMMRYVDSCRQK

SEQ ID NO: 78 - protein, 15 aa, Artificial Sequence
  Description of Artificial Sequence Synthetic peptide
DMMRYVDRYGQLQTQ

SEQ ID NO: 79 - protein, 15 aa, Artificial Sequence
  Description of Artificial Sequence Synthetic peptide
QQPVMALQLFNPEDY

SEQ ID NO: 80 - protein, 15 aa, Artificial Sequence
  Description of Artificial Sequence Synthetic peptide
NLVRDDLPSLTSQEI

SEQ ID NO: 81 - protein, 15 aa, Artificial Sequence
  Description of Artificial Sequence Synthetic peptide
NLVRDDLPALTSQEI

SEQ ID NO: 82 - protein, 15 aa, Artificial Sequence
  Description of Artificial Sequence Synthetic peptide
NLVRDDLPSLTSREI

SEQ ID NO: 83 - protein, 15 aa, Artificial Sequence
  Description of Artificial Sequence Synthetic peptide
DDLPSLTSREIQRRT

SEQ ID NO: 84 - protein, 15 aa, Artificial Sequence
  Description of Artificial Sequence Synthetic peptide
ANQRSAPQWMLPLLL

SEQ ID NO: 85 - protein, 19 aa, Artificial Sequence
  Description of Artificial Sequence Synthetic peptide
HCPCLMCMLKLRHKNRKFL

SEQ ID NO: 86 - protein, 23 aa, Artificial Sequence
  Description of Artificial Sequence Synthetic peptide
ALELQGVVCNYRTKYPDGTIFPK

SEQ ID NO: 87 - protein, 17 aa, Artificial Sequence
  Description of Artificial Sequence Synthetic peptide
KAYLDKNNAYPVECWVP

SEQ ID NO: 88 - protein, 22 aa, Artificial Sequence
  Description of Artificial Sequence Synthetic peptide
LPGDPDMMRYVDRYGQLQTQML

SEQ ID NO: 89 - protein, 19 aa, Artificial Sequence
  Description of Artificial Sequence Synthetic peptide
NLVRDDLPSLTSQEIQRRT

SEQ ID NO: 90 - protein, 15 aa, Artificial Sequence
  Description of Artificial Sequence Synthetic peptide
EYLLYSALTRDPYYI

SEQ ID NO: 91 - protein, 21 aa, Artificial Sequence
  Description of Artificial Sequence Synthetic peptide
SPERKMLPCYSTARIPLPNLY

SEQ ID NO: 92 - protein, 15 aa, Artificial Sequence
  Description of Artificial Sequence Synthetic peptide
EDLFGEPKEPSLSWN

SEQ ID NO: 93 - protein, 15 aa, Artificial Sequence
  Description of Artificial Sequence Synthetic peptide
VYCKDLCCNKFRLVG

SEQ ID NO: 94 - protein, 15 aa, Artificial Sequence
  Description of Artificial Sequence Synthetic peptide
PDIPNQVSECDMLIW

SEQ ID NO: 95 - protein, 15 aa, Artificial Sequence
  Description of Artificial Sequence Synthetic peptide
TGGTPHYATPDWILY

SEQ ID NO: 96 - protein, 15 aa, Artificial Sequence
  Description of Artificial Sequence Synthetic peptide
CKPPYKLLQENKPLL

SEQ ID NO: 97 - protein, 15 aa, Artificial Sequence
  Description of Artificial Sequence Synthetic peptide
PFRHRVSAVNNFCKG

SEQ ID NO: 98 - protein, 15 aa, Artificial Sequence
  Description of Artificial Sequence Synthetic peptide
IVENVPKKRYWVFKG

SEQ ID NO: 99 - protein, 15 aa, Artificial Sequence
  Description of Artificial Sequence Synthetic peptide
SSSQVECTELCCNFP

SEQ ID NO: 100 - protein, 15 aa, Artificial Sequence
  Description of Artificial Sequence Synthetic peptide
CNCFYCFLDKRHKQK

SEQ ID NO: 101 - protein, 15 aa, Artificial Sequence
  Description of Artificial Sequence Synthetic peptide
KQKYKIFRKPPMWIE

SEQ ID NO: 102 - protein, 15 aa, Artificial Sequence
  Description of Artificial Sequence Synthetic peptide
YTLAVVNLPEIPEAL

SEQ ID NO: 103 - protein, 15 aa, Artificial Sequence
  Description of Artificial Sequence Synthetic peptide
MFRQFLQPQKPQVQG

SEQ ID NO: 104 - protein, 15 aa, Artificial Sequence
  Description of Artificial Sequence Synthetic peptide
ANLFGPPVEKQTSKE

SEQ ID NO: 105 - protein, 15 aa, Artificial Sequence
  Description of Artificial Sequence Synthetic peptide
PVEKQTSKEPSKGEL

SEQ ID NO: 106 - protein, 15 aa, Artificial Sequence
  Description of Artificial Sequence Synthetic peptide
ATTGNFQSRGLPYTM

SEQ ID NO: 107 - protein, 15 aa, Artificial Sequence
  Description of Artificial Sequence Synthetic peptide
EDPKDFPSELLSFLS

SEQ ID NO: 108 - protein, 15 aa, Artificial Sequence
  Description of Artificial Sequence Synthetic peptide
FLKCMVYNIPKKRYW

SEQ ID NO: 109 - protein, 15 aa, Artificial Sequence
  Description of Artificial Sequence Synthetic peptide
KRKVEDPKDFPSELL

SEQ ID NO: 110 - protein, 15 aa, Artificial Sequence
  Description of Artificial Sequence Synthetic peptide
SVHDHNQPYHICRGF

SEQ ID NO: 111 - protein, 15 aa, Artificial Sequence
  Description of Artificial Sequence Synthetic peptide
LLCLLRMKHENRKLY

SEQ ID NO: 112 - protein, 15 aa, Artificial Sequence
  Description of Artificial Sequence Synthetic peptide
MKMAPAKRKGSCPGA

SEQ ID NO: 113 - protein, 15 aa, Artificial Sequence
  Description of Artificial Sequence Synthetic peptide
LYRPDDYYDILFPGV

SEQ ID NO: 114 - protein, 15 aa, Artificial Sequence
  Description of Artificial Sequence Synthetic peptide
PLSYSRSSEEAFLEA

SEQ ID NO: 115 - protein, 15 aa, Artificial Sequence
  Description of Artificial Sequence Synthetic peptide
APKKPKEPVQVPKLL

SEQ ID NO: 116 - protein, 15 aa, Artificial Sequence
  Description of Artificial Sequence Synthetic peptide
ARFFRLHFRQRRVKN

SEQ ID NO: 117 - protein, 15 aa, Artificial Sequence
  Description of Artificial Sequence Synthetic peptide
AVGGEPLELQGVLAN

SEQ ID NO: 118 - protein, 15 aa, Artificial Sequence
  Description of Artificial Sequence Synthetic peptide
AVTVQTEVIGITSML

SEQ ID NO: 119 - protein, 15 aa, Artificial Sequence
  Description of Artificial Sequence Synthetic peptide
DKNKAYPVECWVPDP

SEQ ID NO: 120 - protein, 15 aa, Artificial Sequence
  Description of Artificial Sequence Synthetic peptide
DMKVWELYRMETELL

SEQ ID NO: 121 - protein, 15 aa, Artificial Sequence
  Description of Artificial Sequence Synthetic peptide
DMLPCYSVARIPLPN

SEQ ID NO: 122 - protein, 15 aa, Artificial Sequence
  Description of Artificial Sequence Synthetic peptide
DRKMLPCYSTARIPL

SEQ ID NO: 123 - protein, 15 aa, Artificial Sequence
  Description of Artificial Sequence Synthetic peptide
EETPDADTTVCYSLA

SEQ ID NO: 124 - protein, 15 aa, Artificial Sequence
  Description of Artificial Sequence Synthetic peptide
ELLVVPLVNALGNTN

SEQ ID NO: 125 - protein, 15 aa, Artificial Sequence
  Description of Artificial Sequence Synthetic peptide
FFAVGGEPLELQGVL

SEQ ID NO: 126 - protein, 15 aa, Artificial Sequence
  Description of Artificial Sequence Synthetic peptide
FFRLHFRQRRVKNPF

SEQ ID NO: 127 - protein, 15 aa, Artificial Sequence
  Description of Artificial Sequence Synthetic peptide
FLNPQMGNPDEHQKG

SEQ ID NO: 128 - protein, 15 aa, Artificial Sequence
  Description of Artificial Sequence Synthetic peptide
FLTPEMGDPDEHLRG

SEQ ID NO: 129 - protein, 15 aa, Artificial Sequence
  Description of Artificial Sequence Synthetic peptide
GGIEVLGVKTGVDSF

SEQ ID NO: 130 - protein, 15 aa, Artificial Sequence
  Description of Artificial Sequence Synthetic peptide
GGVEVLAAVPLSEET

SEQ ID NO: 131 - protein, 15 aa, Artificial Sequence
  Description of Artificial Sequence Synthetic peptide
KRKGSCPGAAPKKPK

SEQ ID NO: 132 - protein, 15 aa, Artificial Sequence
  Description of Artificial Sequence Synthetic peptide
QVAPPDIPNQVSECD

SEQ ID NO: 133 - protein, 15 aa, Artificial Sequence
  Description of Artificial Sequence Synthetic peptide
RMETELLVVPLVNAL

SEQ ID NO: 134 - protein, 15 aa, Artificial Sequence
  Description of Artificial Sequence Synthetic peptide
RYFKIRLRKRSVKNP

SEQ ID NO: 135 - protein, 15 aa, Artificial Sequence
  Description of Artificial Sequence Synthetic peptide
TFESDSPNRDMLPCY

SEQ ID NO: 136 - protein, 15 aa, Artificial Sequence
  Description of Artificial Sequence Synthetic peptide
TLHVYNSNTPKAKVT

SEQ ID NO: 137 - protein, 15 aa, Artificial Sequence
  Description of Artificial Sequence Synthetic peptide
TSGTQQWKGLPRYFK

SEQ ID NO: 138 - protein, 15 aa, Artificial Sequence
  Description of Artificial Sequence Synthetic peptide
VECFLTPEMGDPDEH

SEQ ID NO: 139 - protein, 15 aa, Artificial Sequence
  Description of Artificial Sequence Synthetic peptide
VMNTEHKAYLDKNKA

SEQ ID NO: 140 - protein, 15 aa, Artificial Sequence
  Description of Artificial Sequence Synthetic peptide
VPLVNALGNTNGVVH

SEQ ID NO: 141 - protein, 15 aa, Artificial Sequence
  Description of Artificial Sequence Synthetic peptide
VQSQVMNTEHKAYLD

SEQ ID NO: 142 - protein, 15 aa, Artificial Sequence
  Description of Artificial Sequence Synthetic peptide
VSECDMKVWELYRME

SEQ ID NO: 143 - protein, 15 aa, Artificial Sequence
  Description of Artificial Sequence Synthetic peptide
WAPDPSRNDNCRYFG

SEQ ID NO: 144 - protein, 15 aa, Artificial Sequence
  Description of Artificial Sequence Synthetic peptide
WELYRMETELLVVPL

SEQ ID NO: 145 - protein, 15 aa, Artificial Sequence
  Description of Artificial Sequence Synthetic peptide
YFGRMVGGAATPPVV

SEQ ID NO: 146 - protein, 15 aa, Artificial Sequence
  Description of Artificial Sequence Synthetic peptide
YFGTLTGGENVPPVL

SEQ ID NO: 147 - protein, 15 aa, Artificial Sequence
  Description of Artificial Sequence Synthetic peptide
YNSNTPKAKVTSERY

SEQ ID NO: 148 - protein, 15 aa, Artificial Sequence
  Description of Artificial Sequence Synthetic peptide
YSTARIPLPNLNEDL

SEQ ID NO: 149 - protein, 15 aa, Artificial Sequence
  Description of Artificial Sequence Synthetic peptide
YSVARIPLPNLNEDL

SEQ ID NO: 150 - protein, 15 aa, Artificial Sequence
  Description of Artificial Sequence Synthetic peptide
CGNLLMREAVTVKTE

SEQ ID NO: 151 - protein, 15 aa, Artificial Sequence
  Description of Artificial Sequence Synthetic peptide
DFSSDSPERKLLPCY

SEQ ID NO: 152 - protein, 15 aa, Artificial Sequence
  Description of Artificial Sequence Synthetic peptide
EHGGGKPIQGSNFHR

SEQ ID NO: 153 - protein, 15 aa, Artificial Sequence
  Description of Artificial Sequence Synthetic peptide
EMGDSDENLRGFSLK

SEQ ID NO: 154 - protein, 15 aa, Artificial Sequence
  Description of Artificial Sequence Synthetic peptide
ENLRGFSLKLSAEYD

SEQ ID NO: 155 - protein, 15 aa, Artificial Sequence
  Description of Artificial Sequence Synthetic peptide
EVECFLNPEMGDSDE

SEQ ID NO: 156 - protein, 15 aa, Artificial Sequence
  Description of Artificial Sequence Synthetic peptide
FLNPEMGDSDENLRG

SEQ ID NO: 157 - protein, 15 aa, Artificial Sequence
  Description of Artificial Sequence Synthetic peptide
IPLPNLYEDLTCGNL

SEQ ID NO: 158 - protein, 15 aa, Artificial Sequence
  Description of Artificial Sequence Synthetic peptide
ITEVECFPNPEMGDP

SEQ ID NO: 159 - protein, 15 aa, Artificial Sequence
  Description of Artificial Sequence Synthetic peptide
KLSAKNDFSSDSPDR

SEQ ID NO: 160 - protein, 15 aa, Artificial Sequence
  Description of Artificial Sequence Synthetic peptide
KMLPCCSTARIPLPN

SEQ ID NO: 161 - protein, 15 aa, Artificial Sequence
  Description of Artificial Sequence Synthetic peptide
KMLPCYGTARIPLPN

SEQ ID NO: 162 - protein, 15 aa, Artificial Sequence
  Description of Artificial Sequence Synthetic peptide
KMLPCYSTTRIPLPN

SEQ ID NO: 163 - protein, 15 aa, Artificial Sequence
  Description of Artificial Sequence Synthetic peptide
KMLPCYSTVRIPLPN

SEQ ID NO: 164 - protein, 15 aa, Artificial Sequence
  Description of Artificial Sequence Synthetic peptide
KPEEPVQVPKLLIKG

SEQ ID NO: 165 - protein, 15 aa, Artificial Sequence
  Description of Artificial Sequence Synthetic peptide
LARYFKTRLRKRSVK

SEQ ID NO: 166 - protein, 15 aa, Artificial Sequence
  Description of Artificial Sequence Synthetic peptide
LARYFRIRLRKRSVK

SEQ ID NO: 167 - protein, 15 aa, Artificial Sequence
  Description of Artificial Sequence Synthetic peptide
EELPGDPDVMRYVDR

SEQ ID NO: 168 - protein, 15 aa, Artificial Sequence
  Description of Artificial Sequence Synthetic peptide
EEVRVFEGTEGLPGD

SEQ ID NO: 169 - protein, 15 aa, Artificial Sequence
  Description of Artificial Sequence Synthetic peptide
EHKAYLDRNKAYPVE

SEQ ID NO: 170 - protein, 15 aa, Artificial Sequence
  Description of Artificial Sequence Synthetic peptide
FFLTDLINRRTPRVD

SEQ ID NO: 171 - protein, 15 aa, Artificial Sequence
  Description of Artificial Sequence Synthetic peptide
FLLADLINRRTPRVD

SEQ ID NO: 172 - protein, 15 aa, Artificial Sequence
  Description of Artificial Sequence Synthetic peptide
FNYGTKYPDGTIFPK

SEQ ID NO: 173 - protein, 15 aa, Artificial Sequence
  Description of Artificial Sequence Synthetic peptide
FNYRTKYPDGTIYPK

SEQ ID NO: 174 - protein, 15 aa, Artificial Sequence
  Description of Artificial Sequence Synthetic peptide
FNYRTRYPDGTIFPK

SEQ ID NO: 175 - protein, 15 aa, Artificial Sequence
  Description of Artificial Sequence Synthetic peptide
FNYRTTYPDGPIFPK

SEQ ID NO: 176 - protein, 15 aa, Artificial Sequence
  Description of Artificial Sequence Synthetic peptide
FNYRTTYPDGTIFPK

SEQ ID NO: 177 - protein, 15 aa, Artificial Sequence
  Description of Artificial Sequence Synthetic peptide
FPLTDLINRRTPRVD

SEQ ID NO: 178 - protein, 15 aa, Artificial Sequence
  Description of Artificial Sequence Synthetic peptide
FRYFKVQLRKRRVKN

SEQ ID NO: 179 - protein, 15 aa, Artificial Sequence
  Description of Artificial Sequence Synthetic peptide
FTKRSGSQQWRGLSR

SEQ ID NO: 180 - protein, 15 aa, Artificial Sequence
  Description of Artificial Sequence Synthetic peptide
GDNLYLSAADVCGMF

SEQ ID NO: 181 - protein, 15 aa, Artificial Sequence
  Description of Artificial Sequence Synthetic peptide
GDNLYLSAVDVCDMF

SEQ ID NO: 182 - protein, 15 aa, Artificial Sequence
  Description of Artificial Sequence Synthetic peptide
GDNLYLSAVDVCGLF

SEQ ID NO: 183 - protein, 15 aa, Artificial Sequence
  Description of Artificial Sequence Synthetic peptide
GDNLYLSAVDVRGMF

SEQ ID NO: 184 - protein, 15 aa, Artificial Sequence
  Description of Artificial Sequence Synthetic peptide
GDNLYLSAVDVYGMF

SEQ ID NO: 185 - protein, 15 aa, Artificial Sequence
  Description of Artificial Sequence Synthetic peptide
GDPDMIRYVDRYGQL

SEQ ID NO: 186 - protein, 15 aa, Artificial Sequence
  Description of Artificial Sequence Synthetic peptide
GDPDMMRYVDRYGQL

SEQ ID NO: 187 - protein, 15 aa, Artificial Sequence
  Description of Artificial Sequence Synthetic peptide
GMFTNKSGSQQWRGL

SEQ ID NO: 188 - protein, 15 aa, Artificial Sequence
  Description of Artificial Sequence Synthetic peptide
IRYVDRYGQLQTKML

SEQ ID NO: 189 - protein, 15 aa, Artificial Sequence
  Description of Artificial Sequence Synthetic peptide
KNATVQSQVMNTDHK

SEQ ID NO: 190 - protein, 15 aa, Artificial Sequence
  Description of Artificial Sequence Synthetic peptide
KVELRKRRVKNPYPI

SEQ ID NO: 191 - protein, 15 aa, Artificial Sequence
  Description of Artificial Sequence Synthetic peptide
KVQLRKRKVKNPYPI

SEQ ID NO: 192 - protein, 15 aa, Artificial Sequence
  Description of Artificial Sequence Synthetic peptide
KVQLRKRRVKDPYPI

SEQ ID NO: 193 - protein, 15 aa, Artificial Sequence
  Description of Artificial Sequence Synthetic peptide
LDEFGGGPLCKGDNL

SEQ ID NO: 194 - protein, 15 aa, Artificial Sequence
  Description of Artificial Sequence Synthetic peptide
LDKNKAYPVECWGPD

SEQ ID NO: 195 - protein, 15 aa, Artificial Sequence
  Description of Artificial Sequence Synthetic peptide
LDKNKAYPVECWVPN

SEQ ID NO: 196 - protein, 15 aa, Artificial Sequence
  Description of Artificial Sequence Synthetic peptide
LINIRTPRVDGQPMY

SEQ ID NO: 197 - protein, 15 aa, Artificial Sequence
  Description of Artificial Sequence Synthetic peptide
LINRRTPGVDGQPMY

SEQ ID NO: 198 - protein, 15 aa, Artificial Sequence
  Description of Artificial Sequence Synthetic peptide
LINRRTPRVNGQPMY

SEQ ID NO: 199 - protein, 15 aa, Artificial Sequence
  Description of Artificial Sequence Synthetic peptide
VARIPLPNINEDLTC

SEQ ID NO: 200 - protein, 15 aa, Artificial Sequence
  Description of Artificial Sequence Synthetic peptide
VARVPLPNLNEDLTC

SEQ ID NO: 201 - protein, 15 aa, Artificial Sequence
  Description of Artificial Sequence Synthetic peptide
VDSCRQKCCNQKPLL

SEQ ID NO: 202 - protein, 15 aa, Artificial Sequence
  Description of Artificial Sequence Synthetic peptide
VECFLTPEMGDPDGH

SEQ ID NO: 203 - protein, 15 aa, Artificial Sequence
  Description of Artificial Sequence Synthetic peptide
VFNYRTKYPDGPIFP

SEQ ID NO: 204 - protein, 15 aa, Artificial Sequence
  Description of Artificial Sequence Synthetic peptide
VGGEALELQGGAFNY

SEQ ID NO: 205 - protein, 15 aa, Artificial Sequence
  Description of Artificial Sequence Synthetic peptide
VGGEALELQGVAFNY

SEQ ID NO: 206 - protein, 15 aa, Artificial Sequence
  Description of Artificial Sequence Synthetic peptide
VGGEALELQGVVCNY

SEQ ID NO: 207 - protein, 15 aa, Artificial Sequence
  Description of Artificial Sequence Synthetic peptide
VGGEALELQGVVFNY

SEQ ID NO: 208 - protein, 15 aa, Artificial Sequence
  Description of Artificial Sequence Synthetic peptide
VKNPYPISFPLTDLI

SEQ ID NO: 209 - protein, 15 aa, Artificial Sequence
  Description of Artificial Sequence Synthetic peptide
VMNTEHKAYLDKNKV

SEQ ID NO: 210 - protein, 15 aa, Artificial Sequence
  Description of Artificial Sequence Synthetic peptide
VMNTEHKAYLDRNKA

SEQ ID NO: 211 - protein, 15 aa, Artificial Sequence
  Description of Artificial Sequence Synthetic peptide
VVNTEHKAYLDKNKA

SEQ ID NO: 212 - protein, 15 aa, Artificial Sequence
  Description of Artificial Sequence Synthetic peptide
WRGLSRYFKVQPRKR

SEQ ID NO: 213 - protein, 15 aa, Artificial Sequence
  Description of Artificial Sequence Synthetic peptide
WRGLSRYFRVQLRKR

SEQ ID NO: 214 - protein, 15 aa, Artificial Sequence
  Description of Artificial Sequence Synthetic peptide
YLDKNKVYPVECWVP

SEQ ID NO: 215 - protein, 15 aa, Artificial Sequence
  Description of Artificial Sequence Synthetic peptide
YLSAVDVCGMFTDRS

SEQ ID NO: 216 - protein, 15 aa, Artificial Sequence
  Description of Artificial Sequence Synthetic peptide
YPISFLLADLINRRT

SEQ ID NO: 217 - protein, 15 aa, Artificial Sequence
  Description of Artificial Sequence Synthetic peptide
YPISFPLTDLINRRT

SEQ ID NO: 218 - protein, 15 aa, Artificial Sequence
  Description of Artificial Sequence Synthetic peptide
YPITFLLTDLINRRT

SEQ ID NO: 219 - protein, 15 aa, Artificial Sequence
  Description of Artificial Sequence Synthetic peptide
KVTSERYSVEWAPDP

SEQ ID NO: 220 - protein, 15 aa, Artificial Sequence
  Description of Artificial Sequence Synthetic peptide
LWLQGRLYITCADML

SEQ ID NO: 221 - protein, 15 aa, Artificial Sequence
  Description of Artificial Sequence Synthetic peptide
MSCTACRPQKRLTRP

SEQ ID NO: 222 - protein, 15 aa, Artificial Sequence
  Description of Artificial Sequence Synthetic peptide
QLPRTVTLQSQAPLL

SEQ ID NO: 223 - protein, 15 aa, Artificial Sequence
  Description of Artificial Sequence Synthetic peptide
RMETELLVVPLVNAG

SEQ ID NO: 224 - protein, 15 aa, Artificial Sequence
  Description of Artificial Sequence Synthetic peptide
VVRGAATPPDVSYGN

SEQ ID NO: 225 - protein, 15 aa, Artificial Sequence
  Description of Artificial Sequence Synthetic peptide
YSISSAIHDKESGSI

SEQ ID NO: 226 - protein, 15 aa, Artificial Sequence
  Description of Artificial Sequence Synthetic peptide
CDTLQMWEAISVKTE

SEQ ID NO: 227 - protein, 15 aa, Artificial Sequence
  Description of Artificial Sequence Synthetic peptide
EPLPGDPDIVRFLDK

SEQ ID NO: 228 - protein, 15 aa, Artificial Sequence
  Description of Artificial Sequence Synthetic peptide
EVRIYEGSEPLPGDP

SEQ ID NO: 229 - protein, 15 aa, Artificial Sequence
  Description of Artificial Sequence Synthetic peptide
GAGIPVSGVNYHMFA

SEQ ID NO: 230 - protein, 15 aa, Artificial Sequence
  Description of Artificial Sequence Synthetic peptide
GKAPLKGPQKASQKE

SEQ ID NO: 231 - protein, 15 aa, Artificial Sequence
  Description of Artificial Sequence Synthetic peptide
GKAPLKGPQQASQKE

SEQ ID NO: 232 - protein, 15 aa, Artificial Sequence
  Description of Artificial Sequence Synthetic peptide
SDNPNATTLPTYSVA

SEQ ID NO: 233 - protein, 15 aa, Artificial Sequence
  Description of Artificial Sequence Synthetic peptide
SGLMPQIQGQPMEGT

SEQ ID NO: 234 - protein, 15 aa, Artificial Sequence
  Description of Artificial Sequence Synthetic peptide
VQGTTLHMFSVGGEP

SEQ ID NO: 235 - protein, 15 aa, Artificial Sequence
  Description of Artificial Sequence Synthetic peptide
VSVKTEVMGISSLVN

SEQ ID NO: 236 - protein, 15 aa, Artificial Sequence
  Description of Artificial Sequence Synthetic peptide
YPTDMVTIKNMKPVN

SEQ ID NO: 237 - protein, 15 aa, Artificial Sequence
  Description of Artificial Sequence Synthetic peptide
YSESQNWRGLPRYFN

SEQ ID NO: 238 - protein, 15 aa, Artificial Sequence
  Description of Artificial Sequence Synthetic peptide
ENTRYYGSYTGGQST

SEQ ID NO: 239 - protein, 15 aa, Artificial Sequence
  Description of Artificial Sequence Synthetic peptide
GEPLELQFLTGNYRT

SEQ ID NO: 240 - protein, 15 aa, Artificial Sequence
  Description of Artificial Sequence Synthetic peptide
GLPRYFNILLRKRTV

SEQ ID NO: 241 - protein, 15 aa, Artificial Sequence
  Description of Artificial Sequence Synthetic peptide
GTEGLPGDPDMVRYI

SEQ ID NO: 242 - protein, 15 aa, Artificial Sequence
  Description of Artificial Sequence Synthetic peptide
GVSSLVNVHMATKRM

SEQ ID NO: 243 - protein, 15 aa, Artificial Sequence
  Description of Artificial Sequence Synthetic peptide
HMATKRMYDDKGIGF

SEQ ID NO: 244 - protein, 15 aa, Artificial Sequence
  Description of Artificial Sequence Synthetic peptide
IELYLNTRMGQNDES

SEQ ID NO: 245 - protein, 15 aa, Artificial Sequence
  Description of Artificial Sequence Synthetic peptide
IGFPVEGMNFHMFAV

SEQ ID NO: 246 - protein, 15 aa, Artificial Sequence
  Description of Artificial Sequence Synthetic peptide
KDGDMQYRGLPRYFN

SEQ ID NO: 247 - protein, 15 aa, Artificial Sequence
  Description of Artificial Sequence Synthetic peptide
KFGQDKTRPPFPARL

SEQ ID NO: 248 - protein, 15 aa, Artificial Sequence
  Description of Artificial Sequence Synthetic peptide
KQKLTKDGAFPVECW

SEQ ID NO: 249 - protein, 15 aa, Artificial Sequence
  Description of Artificial Sequence Synthetic peptide
LPGDPDMVRYIDKFG

SEQ ID NO: 250 - protein, 15 aa, Artificial Sequence
  Description of Artificial Sequence Synthetic peptide
LSTQVEEVRVYDGTE

SEQ ID NO: 251 - protein, 15 aa, Artificial Sequence
  Description of Artificial Sequence Synthetic peptide
LVNVHMATKRMYDDK

SEQ ID NO: 252 - protein, 15 aa, Artificial Sequence
  Description of Artificial Sequence Synthetic peptide
PDMVRYIDKFGQDKT

SEQ ID NO: 253 - protein, 15 aa, Artificial Sequence
  Description of Artificial Sequence Synthetic peptide
PVLQFTNTVTTVLLD

SEQ ID NO: 254 - protein, 15 aa, Artificial Sequence
  Description of Artificial Sequence Synthetic peptide
QSTPPVLQFTNTVTT

SEQ ID NO: 255 - protein, 15 aa, Artificial Sequence
  Description of Artificial Sequence Synthetic peptide
RTVRNPYPVSSLLNN

SEQ ID NO: 256 - protein, 15 aa, Artificial Sequence
  Description of Artificial Sequence Synthetic peptide
RYIDKFGQDKTRPPF

SEQ ID NO: 257 - protein, 15 aa, Artificial Sequence
  Description of Artificial Sequence Synthetic peptide
TEVVGVSSLVNVHMA

SEQ ID NO: 258 - protein, 15 aa, Artificial Sequence
  Description of Artificial Sequence Synthetic peptide
TKDGAFPVECWCPDP

SEQ ID NO: 259 - protein, 15 aa, Artificial Sequence
  Description of Artificial Sequence Synthetic peptide
TQGLNPHYKQKLTKD

SEQ ID NO: 260 - protein, 15 aa, Artificial Sequence
  Description of Artificial Sequence Synthetic peptide
VEGMNFHMFAVGGEP

SEQ ID NO: 261 - protein, 15 aa, Artificial Sequence
  Description of Artificial Sequence Synthetic peptide
VSVKTEVVGVSSLVN

SEQ ID NO: 262 - protein, 15 aa, Artificial Sequence
  Description of Artificial Sequence Synthetic peptide
YRTDYSANDKLVVPP

SEQ ID NO: 263 - protein, 15 aa, Artificial Sequence
  Description of Artificial Sequence Synthetic peptide
YYGSYTGGQSTPPVL

SEQ ID NO: 264 - protein, 15 aa, Artificial Sequence
  Description of Artificial Sequence Synthetic peptide
CGGSPLDVIGINPDP

SEQ ID NO: 265 - protein, 15 aa, Artificial Sequence
  Description of Artificial Sequence Synthetic peptide
EDTIYKVEAILLPNF

SEQ ID NO: 266 - protein, 15 aa, Artificial Sequence
  Description of Artificial Sequence Synthetic peptide
ETELIFTPQVGSAGY

SEQ ID NO: 267 - protein, 15 aa, Artificial Sequence
  Description of Artificial Sequence Synthetic peptide
FLQPQKPQVQGTQPN

SEQ ID NO: 268 - protein, 15 aa, Artificial Sequence
  Description of Artificial Sequence Synthetic peptide
GLAALGGITEGAALL

SEQ ID NO: 269 - protein, 15 aa, Artificial Sequence
  Description of Artificial Sequence Synthetic peptide
KRKQDELHPVSPTKK

SEQ ID NO: 270 - protein, 15 aa, Artificial Sequence
  Description of Artificial Sequence Synthetic peptide
LPELPSLQDVFNRIA

SEQ ID NO: 271 - protein, 15 aa, Artificial Sequence
  Description of Artificial Sequence Synthetic peptide
LVASYLPELPSLQDV

SEQ ID NO: 272 - protein, 15 aa, Artificial Sequence
  Description of Artificial Sequence Synthetic peptide
MALVPIPEYQLETGI

SEQ ID NO: 273 - protein, 15 aa, Artificial Sequence
  Description of Artificial Sequence Synthetic peptide
PIPEYQLETGIPGIP

SEQ ID NO: 274 - protein, 15 aa, Artificial Sequence
  Description of Artificial Sequence Synthetic peptide
PSLQDVFNRIAFGIW

SEQ ID NO: 275 - protein, 15 aa, Artificial Sequence
  Description of Artificial Sequence Synthetic peptide
PVNAIATQVRSLATT

SEQ ID NO: 276 - protein, 15 aa, Artificial Sequence
  Description of Artificial Sequence Synthetic peptide
VHKPIHAPYSGMALV

SEQ ID NO: 277 - protein, 15 aa, Artificial Sequence
  Description of Artificial Sequence Synthetic peptide
VLSDEIQRLLRDLEY

SEQ ID NO: 278 - protein, 15 aa, Artificial Sequence
  Description of Artificial Sequence Synthetic peptide
HIGGTLQQQTPDWLL

SEQ ID NO: 279 - protein, 15 aa, Artificial Sequence
  Description of Artificial Sequence Synthetic peptide
LDPLQWENSLLHSVG

SEQ ID NO: 280 - protein, 15 aa, Artificial Sequence
  Description of Artificial Sequence Synthetic peptide
QWENSLLHSVGQDIF

SEQ ID NO: 281 - protein, 15 aa, Artificial Sequence
  Description of Artificial Sequence Synthetic peptide
RHALMAFSLDPLQWE

SEQ ID NO: 282 - protein, 15 aa, Artificial Sequence
  Description of Artificial Sequence Synthetic peptide
TLQQQTPDWLLPLVL

SEQ ID NO: 283 - protein, 15 aa, Artificial Sequence
  Description of Artificial Sequence Synthetic peptide
ADSIQQVTERWEAQS

SEQ ID NO: 284 - protein, 15 aa, Artificial Sequence
  Description of Artificial Sequence Synthetic peptide
APQWMLPLLLGLYGS

SEQ ID NO: 285 - protein, 15 aa, Artificial Sequence
  Description of Artificial Sequence Synthetic peptide
DDYYDILFPGVQTFV

SEQ ID NO: 286 - protein, 15 aa, Artificial Sequence
  Description of Artificial Sequence Synthetic peptide
KAYEDGPNKKKRKLS

SEQ ID NO: 287 - protein, 15 aa, Artificial Sequence
  Description of Artificial Sequence Synthetic peptide
PGVQTFVHSVQYLDP

SEQ ID NO: 288 - protein, 15 aa, Artificial Sequence
  Description of Artificial Sequence Synthetic peptide
QDYYSTLSPIRPTMV

SEQ ID NO: 289 - protein, 15 aa, Artificial Sequence
  Description of Artificial Sequence Synthetic peptide
SVQYLDPRHWGPTLF

SEQ ID NO: 290 - protein, 15 aa, Artificial Sequence
  Description of Artificial Sequence Synthetic peptide
TTWTVINAPVNWYNS

SEQ ID NO: 291 - protein, 15 aa, Artificial Sequence
  Description of Artificial Sequence Synthetic peptide
DVFNRIAYGIWTSYY

SEQ ID NO: 292 - protein, 15 aa, Artificial Sequence
  Description of Artificial Sequence Synthetic peptide
ELQRLLGDLEYGFRT

SEQ ID NO: 293 - protein, 15 aa, Artificial Sequence
  Description of Artificial Sequence Synthetic peptide
FIASHLPELPSLQDV

SEQ ID NO: 294 - protein, 15 aa, Artificial Sequence
  Description of Artificial Sequence Synthetic peptide
GGIYTALAADRPGDL

SEQ ID NO: 295 - protein, 15 aa, Artificial Sequence
  Description of Artificial Sequence Synthetic peptide
GIWTSYYNTGRTVVN

SEQ ID NO: 296 - protein, 15 aa, Artificial Sequence
  Description of Artificial Sequence Synthetic peptide
GLAALGGLTESAALL

SEQ ID NO: 297 - protein, 15 aa, Artificial Sequence
  Description of Artificial Sequence Synthetic peptide
LLGDLEYGFRTALAT

SEQ ID NO: 298 - protein, 15 aa, Artificial Sequence
  Description of Artificial Sequence Synthetic peptide
MALAPIPEYNLETGI

SEQ ID NO: 299 - protein, 15 aa, Artificial Sequence
  Description of Artificial Sequence Synthetic peptide
PDWILYVLEELNSDI

SEQ ID NO: 300 - protein, 15 aa, Artificial Sequence
  Description of Artificial Sequence Synthetic peptide
PIPEYNLETGIPGVP

SEQ ID NO: 301 - protein, 15 aa, Artificial Sequence
  Description of Artificial Sequence Synthetic peptide
PSLQDVFNRIAYGIW

SEQ ID NO: 302 - protein, 15 aa, Artificial Sequence
  Description of Artificial Sequence Synthetic peptide
CGGKSLNVNMPLERL

SEQ ID NO: 303 - protein, 15 aa, Artificial Sequence
  Description of Artificial Sequence Synthetic peptide
FLKCIVLNIPKKRYW

SEQ ID NO: 304 - protein, 15 aa, Artificial Sequence
  Description of Artificial Sequence Synthetic peptide
GNIPVMRKAYLKKCK

SEQ ID NO: 305 - protein, 15 aa, Artificial Sequence
  Description of Artificial Sequence Synthetic peptide
HFNHHEKHYYNAQIF

SEQ ID NO: 306 - protein, 15 aa, Artificial Sequence
  Description of Artificial Sequence Synthetic peptide
ISNLDCLRDYLDGSV

SEQ ID NO: 307 - protein, 15 aa, Artificial Sequence
  Description of Artificial Sequence Synthetic peptide
KGFQCFKKPKTPPPK

SEQ ID NO: 308 - protein, 15 aa, Artificial Sequence
  Description of Artificial Sequence Synthetic peptide
LLDLCGGKSLNVNMP

SEQ ID NO: 309 - protein, 15 aa, Artificial Sequence
  Description of Artificial Sequence Synthetic peptide
LMDLLGLDRSAWGNI

SEQ ID NO: 310 - protein, 15 aa, Artificial Sequence
  Description of Artificial Sequence Synthetic peptide
MKANVGMGRPILDFP

SEQ ID NO: 311 - protein, 15 aa, Artificial Sequence
  Description of Artificial Sequence Synthetic peptide
RKHQNKRTQVFPPGI

SEQ ID NO: 312 - protein, 15 aa, Artificial Sequence
  Description of Artificial Sequence Synthetic peptide
RVSAINNYCQKLCTF

SEQ ID NO: 313 - protein, 15 aa, Artificial Sequence
  Description of Artificial Sequence Synthetic peptide
SGHGISNLDCLRDYL

SEQ ID NO: 314 - protein, 15 aa, Artificial Sequence
  Description of Artificial Sequence Synthetic peptide
TKCEDVFLLMGMYLD

SEQ ID NO: 315 - protein, 15 aa, Artificial Sequence
  Description of Artificial Sequence Synthetic peptide
VDSIHMTREEMLVER

SEQ ID NO: 316 - protein, 15 aa, Artificial Sequence
  Description of Artificial Sequence Synthetic peptide
VGMGRPILDFPREED

SEQ ID NO: 317 - protein, 15 aa, Artificial Sequence
  Description of Artificial Sequence Synthetic peptide
VNLERKHQNKRTQVF

SEQ ID NO: 318 - protein, 15 aa, Artificial Sequence
  Description of Artificial Sequence Synthetic peptide
VPTYGTDEWESWWNT

SEQ ID NO: 319 - protein, 15 aa, Artificial Sequence
  Description of Artificial Sequence Synthetic peptide
WESWWNTFNEKWDED

SEQ ID NO: 320 - protein, 15 aa, Artificial Sequence
  Description of Artificial Sequence Synthetic peptide
WNTFNEKWDEDLFCH

SEQ ID NO: 321 - protein, 15 aa, Artificial Sequence
  Description of Artificial Sequence Synthetic peptide
ALDQYMVVFEDVKGQ

SEQ ID NO: 322 - protein, 15 aa, Artificial Sequence
  Description of Artificial Sequence Synthetic peptide
ALEFDIDDVYYLLGS

SEQ ID NO: 323 - protein, 15 aa, Artificial Sequence
  Description of Artificial Sequence Synthetic peptide
CSQATPPKKKHAFDA

SEQ ID NO: 324 - protein, 15 aa, Artificial Sequence
  Description of Artificial Sequence Synthetic peptide
EFVSHAVFSNKCITC

SEQ ID NO: 325 - protein, 15 aa, Artificial Sequence
  Description of Artificial Sequence Synthetic peptide
EIQSNVVYWKEVLDN

SEQ ID NO: 326 - protein, 15 aa, Artificial Sequence
  Description of Artificial Sequence Synthetic peptide
ELGVALDQYMVVFED

SEQ ID NO: 327 - protein, 15 aa, Artificial Sequence
  Description of Artificial Sequence Synthetic peptide
FFLTPHKHRVSAINN

SEQ ID NO: 328 - protein, 15 aa, Artificial Sequence
  Description of Artificial Sequence Synthetic peptide
FLFCKGVNNPYGLYS

SEQ ID NO: 329 - protein, 15 aa, Artificial Sequence
  Description of Artificial Sequence Synthetic peptide
GEPKEPSLSWNQIAN

SEQ ID NO: 330 - protein, 15 aa, Artificial Sequence
  Description of Artificial Sequence Synthetic peptide
GNGVNNLDNLRDYLD

SEQ ID NO: 331 - protein, 15 aa, Artificial Sequence
  Description of Artificial Sequence Synthetic peptide
HKRVHVQNHENAVLL

SEQ ID NO: 332 - protein, 15 aa, Artificial Sequence
  Description of Artificial Sequence Synthetic peptide
IPGGLKENEFNPEDL

SEQ ID NO: 333 - protein, 15 aa, Artificial Sequence
  Description of Artificial Sequence Synthetic peptide
LDNYIGLTEFATMQM

SEQ ID NO: 334 - protein, 15 aa, Artificial Sequence
  Description of Artificial Sequence Synthetic peptide
LKENEFNPEDLFGEP

SEQ ID NO: 335 - protein, 15 aa, Artificial Sequence
  Description of Artificial Sequence Synthetic peptide
TSDKAIELYDKIEKF

SEQ ID NO: 336 - protein, 15 aa, Artificial Sequence
  Description of Artificial Sequence Synthetic peptide
VDFKSRHACELGCIL

SEQ ID NO: 337 - protein, 15 aa, Artificial Sequence
  Description of Artificial Sequence Synthetic peptide
YKLLQENKPLLNYEF

SEQ ID NO: 338 - protein, 15 aa, Artificial Sequence
  Description of Artificial Sequence Synthetic peptide
YRSSSFTTPKTPPPF

SEQ ID NO: 339 - protein, 15 aa, Artificial Sequence
  Description of Artificial Sequence Synthetic peptide
AWLHCLLPKMDSVVY

SEQ ID NO: 340 - protein, 15 aa, Artificial Sequence
  Description of Artificial Sequence Synthetic peptide
CLLPKMDSVVYDFLK

SEQ ID NO: 341 - protein, 15 aa, Artificial Sequence
  Description of Artificial Sequence Synthetic peptide
EFAQSIQSRIVEWKE

SEQ ID NO: 342 - protein, 15 aa, Artificial Sequence
  Description of Artificial Sequence Synthetic peptide
EKMKKMNTLYKKMED

SEQ ID NO: 343 - protein, 15 aa, Artificial Sequence
  Description of Artificial Sequence Synthetic peptide
ELGVAIDQFLVVFED

SEQ ID NO: 344 - protein, 15 aa, Artificial Sequence
  Description of Artificial Sequence Synthetic peptide
EYLMYSALTRDPFSV

SEQ ID NO: 345 - protein, 15 aa, Artificial Sequence
  Description of Artificial Sequence Synthetic peptide
FGGFWDATEIPTYGT

SEQ ID NO: 346 - protein, 15 aa, Artificial Sequence
  Description of Artificial Sequence Synthetic peptide
FGSTGSADIEEWMAG

SEQ ID NO: 347 - protein, 15 aa, Artificial Sequence
  Description of Artificial Sequence Synthetic peptide
GNIPLMRKAYLKKCK

SEQ ID NO: 348 - protein, 15 aa, Artificial Sequence
  Description of Artificial Sequence Synthetic peptide
GQGINNLDNLRDYLD

SEQ ID NO: 349 - protein, 15 aa, Artificial Sequence
  Description of Artificial Sequence Synthetic peptide
KEKAALLYKKIMEKY

SEQ ID NO: 350 - protein, 15 aa, Artificial Sequence
  Description of Artificial Sequence Synthetic peptide
KMNTLYKKMEDGVKY

SEQ ID NO: 351 - protein, 15 aa, Artificial Sequence
  Description of Artificial Sequence Synthetic peptide
LMRKAYLKKCKEFHP

SEQ ID NO: 352 - protein, 15 aa, Artificial Sequence
  Description of Artificial Sequence Synthetic peptide
MDKVLNREESLQLMD

SEQ ID NO: 353 - protein, 15 aa, Artificial Sequence
  Description of Artificial Sequence Synthetic peptide
MLTNRFNDLLDRMDI

SEQ ID NO: 354 - protein, 15 aa, Artificial Sequence
  Description of Artificial Sequence Synthetic peptide
RVSAINNYAQKLCTF

SEQ ID NO: 355 - protein, 15 aa, Artificial Sequence
  Description of Artificial Sequence Synthetic peptide
SFQAPQPSQSSQSVH

SEQ ID NO: 356 - protein, 15 aa, Artificial Sequence
  Description of Artificial Sequence Synthetic peptide
TREQMLTNRFNDLLD

SEQ ID NO: 357 - protein, 15 aa, Artificial Sequence
  Description of Artificial Sequence Synthetic peptide
VKYAHQPDFGGFWDA

SEQ ID NO: 358 - protein, 15 aa, Artificial Sequence
  Description of Artificial Sequence Synthetic peptide
VTEYAMETKCDDVLL

SEQ ID NO: 359 - protein, 15 aa, Artificial Sequence
  Description of Artificial Sequence Synthetic peptide
WDATEIPTYGTDEWE

SEQ ID NO: 360 - protein, 15 aa, Artificial Sequence
  Description of Artificial Sequence Synthetic peptide
YHICRGFTCFKKPPT

SEQ ID NO: 361 - protein, 15 aa, Artificial Sequence
  Description of Artificial Sequence Synthetic peptide
AAALLDLCGGKALNI

SEQ ID NO: 362 - protein, 15 aa, Artificial Sequence
  Description of Artificial Sequence Synthetic peptide
AWYLGLNGKIDELVY

SEQ ID NO: 363 - protein, 15 aa, Artificial Sequence
  Description of Artificial Sequence Synthetic peptide
CVSTVHQLNEEEDEV

SEQ ID NO: 364 - protein, 15 aa, Artificial Sequence
  Description of Artificial Sequence Synthetic peptide
GVNKEYLLYSALTRE

SEQ ID NO: 365 - protein, 15 aa, Artificial Sequence
  Description of Artificial Sequence Synthetic peptide
HMTREEMLVQRFNFL

SEQ ID NO: 366 - protein, 15 aa, Artificial Sequence
  Description of Artificial Sequence Synthetic peptide
KRVDSLHMTREEMLT

SEQ ID NO: 367 - protein, 15 aa, Artificial Sequence
  Description of Artificial Sequence Synthetic peptide
LGLDRSAWGNIPIMR

SEQ ID NO: 368 - protein, 15 aa, Artificial Sequence
  Description of Artificial Sequence Synthetic peptide
MLTDRFNHILDKMDL

SEQ ID NO: 369 - protein, 15 aa, Artificial Sequence
  Description of Artificial Sequence Synthetic peptide
SLHMTREEMLTDRFN

SEQ ID NO: 370 - protein, 15 aa, Artificial Sequence
  Description of Artificial Sequence Synthetic peptide
VDSIHMTREEMLVQR

SEQ ID NO: 371 - protein, 15 aa, Artificial Sequence
  Description of Artificial Sequence Synthetic peptide
YLRKSLSSSEYLLEK

SEQ ID NO: 372 - protein, 15 aa, Artificial Sequence
  Description of Artificial Sequence Synthetic peptide
CADFPLCPDTLYCKE

SEQ ID NO: 373 - protein, 15 aa, Artificial Sequence
  Description of Artificial Sequence Synthetic peptide
LRHLNRKFLRKEPLV

SEQ ID NO: 374 - protein, 15 aa, Artificial Sequence
  Description of Artificial Sequence Synthetic peptide
PDFGTWSSSEVCADF

SEQ ID NO: 375 - protein, 15 aa, Artificial Sequence
  Description of Artificial Sequence Synthetic peptide
SSEVCADFPLCPDTL

SEQ ID NO: 376 - protein, 15 aa, Artificial Sequence
  Description of Artificial Sequence Synthetic peptide
CDFPPNSDTLYCKEW

SEQ ID NO: 377 - protein, 15 aa, Artificial Sequence
  Description of Artificial Sequence Synthetic peptide
NCATNPSVHCPCLMC

SEQ ID NO: 378 - protein, 15 aa, Artificial Sequence
  Description of Artificial Sequence Synthetic peptide
CYREWFFFPISMQTF

SEQ ID NO: 379 - protein, 15 aa, Artificial Sequence
  Description of Artificial Sequence Synthetic peptide
FWKVIIFNTEIRAVQ

SEQ ID NO: 380 - protein, 15 aa, Artificial Sequence
  Description of Artificial Sequence Synthetic peptide
WIECYCYKCYREWFF

SEQ ID NO: 381 - protein, 15 aa, Artificial Sequence
  Description of Artificial Sequence Synthetic peptide
YCYKCYREWFFFPIS

SEQ ID NO: 382 - protein, 15 aa, Artificial Sequence
  Description of Artificial Sequence Synthetic peptide
YIMKQWDVCIHGYNH

SEQ ID NO: 383 - protein, 15 aa, Artificial Sequence
  Description of Artificial Sequence Synthetic peptide
YYEAYIMKQWDVCIH

SEQ ID NO: 384 - protein, 15 aa, Artificial Sequence
  Description of Artificial Sequence Synthetic peptide
FGFPPTWESFDWWQK

SEQ ID NO: 385 - protein, 15 aa, Artificial Sequence
  Description of Artificial Sequence Synthetic peptide
FSMFDEVSTKFPWEE

SEQ ID NO: 386 - protein, 15 aa, Artificial Sequence
  Description of Artificial Sequence Synthetic peptide
GTLKDYMQSGYNARF

SEQ ID NO: 387 - protein, 15 aa, Artificial Sequence
  Description of Artificial Sequence Synthetic peptide
RGPGCMLKQLRDSKC

SEQ ID NO: 388 - protein, 15 aa, Artificial Sequence
  Description of Artificial Sequence Synthetic peptide
LDKDNAYPVECWVPD

SEQ ID NO: 389 - protein, 15 aa, Artificial Sequence
  Description of Artificial Sequence Synthetic peptide
LDKNNAYPVECWIPD

SEQ ID NO: 390 - protein, 15 aa, Artificial Sequence
  Description of Artificial Sequence Synthetic peptide
LDKNNAYPVECWVPD

SEQ ID NO: 391 - protein, 15 aa, Artificial Sequence
  Description of Artificial Sequence Synthetic peptide
LELQGVLANYRTKYP

SEQ ID NO: 392 - protein, 15 aa, Artificial Sequence
  Description of Artificial Sequence Synthetic peptide
LMNYRSKYPDGTITP

SEQ ID NO: 393 - protein, 15 aa, Artificial Sequence
  Description of Artificial Sequence Synthetic peptide
LPATVTLQATGPILN

SEQ ID NO: 394 - protein, 15 aa, Artificial Sequence
  Description of Artificial Sequence Synthetic peptide
LPGDPDMIRYIDKQG

SEQ ID NO: 395 - protein, 15 aa, Artificial Sequence
  Description of Artificial Sequence Synthetic peptide
LPGDPDMIRYIDRQG

SEQ ID NO: 396 - protein, 15 aa, Artificial Sequence
  Description of Artificial Sequence Synthetic peptide
LPGDPDMMRYVDKYG

SEQ ID NO: 397 - protein, 15 aa, Artificial Sequence
  Description of Artificial Sequence Synthetic peptide
LSDLINRRTQRVDGQ

SEQ ID NO: 398 - protein, 15 aa, Artificial Sequence
  Description of Artificial Sequence Synthetic peptide
MESQVEEVRVFDGTE

SEQ ID NO: 399 - protein, 15 aa, Artificial Sequence
  Description of Artificial Sequence Synthetic peptide
MQGVLMNYRSKYPDG

SEQ ID NO: 400 - protein, 15 aa, Artificial Sequence
  Description of Artificial Sequence Synthetic peptide
MSCTPCRPQKRLTRP

SEQ ID NO: 401 - protein, 15 aa, Artificial Sequence
  Description of Artificial Sequence Synthetic peptide
NQVSECDMLIWELYR

SEQ ID NO: 402 - protein, 15 aa, Artificial Sequence
  Description of Artificial Sequence Synthetic peptide
PDMIRYIDKQGQLQT

SEQ ID NO: 403 - protein, 15 aa, Artificial Sequence
  Description of Artificial Sequence Synthetic peptide
PYPISFLLSDLINRR

SEQ ID NO: 404 - protein, 15 aa, Artificial Sequence
  Description of Artificial Sequence Synthetic peptide
QLPRTVTLQSQTPLL

SEQ ID NO: 405 - protein, 15 aa, Artificial Sequence
  Description of Artificial Sequence Synthetic peptide
LMREAVTVKTEVMGI

SEQ ID NO: 406 - protein, 15 aa, Artificial Sequence
  Description of Artificial Sequence Synthetic peptide
LTCGNLLMWEAVTLQ

SEQ ID NO: 407 - protein, 15 aa, Artificial Sequence
  Description of Artificial Sequence Synthetic peptide
MLPCYSAARIPLPNL

SEQ ID NO: 408 - protein, 15 aa, Artificial Sequence
  Description of Artificial Sequence Synthetic peptide
MWEAATVKTEVIGIT

SEQ ID NO: 409 - protein, 15 aa, Artificial Sequence
  Description of Artificial Sequence Synthetic peptide
MWEAVQVQTEVIGIT

SEQ ID NO: 410 - protein, 15 aa, Artificial Sequence
  Description of Artificial Sequence Synthetic peptide
NLLMWEAVTVQTEVT

SEQ ID NO: 411 - protein, 15 aa, Artificial Sequence
  Description of Artificial Sequence Synthetic peptide
PLEMQGVLLNYRTKY

SEQ ID NO: 412 - protein, 15 aa, Artificial Sequence
  Description of Artificial Sequence Synthetic peptide
PLEMQGVLMNYWTKY

SEQ ID NO: 413 - protein, 15 aa, Artificial Sequence
  Description of Artificial Sequence Synthetic peptide
PNLNEDLTCENLLMW

SEQ ID NO: 414 - protein, 15 aa, Artificial Sequence
  Description of Artificial Sequence Synthetic peptide
PNLNEDLTCGNLLMR

SEQ ID NO: 415 - protein, 15 aa, Artificial Sequence
  Description of Artificial Sequence Synthetic peptide
PNLNEDLTCGNLLVW

SEQ ID NO: 416 - protein, 15 aa, Artificial Sequence
  Description of Artificial Sequence Synthetic peptide
PNLNEDLTRGNLLMW

SEQ ID NO: 417 - protein, 15 aa, Artificial Sequence
  Description of Artificial Sequence Synthetic peptide
PQRKMLPCYSTARIP

SEQ ID NO: 418 - protein, 15 aa, Artificial Sequence
  Description of Artificial Sequence Synthetic peptide
PYPISFSLSDLINRR

SEQ ID NO: 419 - protein, 15 aa, Artificial Sequence
  Description of Artificial Sequence Synthetic peptide
RIPLPNLNEDLTCEN

SEQ ID NO: 420 - protein, 15 aa, Artificial Sequence
  Description of Artificial Sequence Synthetic peptide
SFLLSDLITRRTQRV

SEQ ID NO: 421 - protein, 15 aa, Artificial Sequence
  Description of Artificial Sequence Synthetic peptide
SPERKMLPCYGTARI

SEQ ID NO: 422 - protein, 15 aa, Artificial Sequence
  Description of Artificial Sequence Synthetic peptide
TKYPHGTITPKNPTV

SEQ ID NO: 423 - protein, 15 aa, Artificial Sequence
  Description of Artificial Sequence Synthetic peptide
VSAADICGLFINSSG

SEQ ID NO: 424 - protein, 15 aa, Artificial Sequence
  Description of Artificial Sequence Synthetic peptide
YSAARIPLPNLNEDL

SEQ ID NO: 425 - protein, 15 aa, Artificial Sequence
  Description of Artificial Sequence Synthetic peptide
YSLKLTAENAFDSDS

SEQ ID NO: 426 - protein, 15 aa, Artificial Sequence
  Description of Artificial Sequence Synthetic peptide
ALELQGVVFNYGTKY

SEQ ID NO: 427 - protein, 15 aa, Artificial Sequence
  Description of Artificial Sequence Synthetic peptide
ARIPLPILNEDLTCG

SEQ ID NO: 428 - protein, 15 aa, Artificial Sequence
  Description of Artificial Sequence Synthetic peptide
CGNIPMWEAVTLKTE

SEQ ID NO: 429 - protein, 15 aa, Artificial Sequence
  Description of Artificial Sequence Synthetic peptide
CWVPDPTRNENPRYF

SEQ ID NO: 430 - protein, 15 aa, Artificial Sequence
  Description of Artificial Sequence Synthetic peptide
DEFGVGLLCKGDNLY

SEQ ID NO: 431 - protein, 15 aa, Artificial Sequence
  Description of Artificial Sequence Synthetic peptide
DKTKAYPVECWVPDP

SEQ ID NO: 432 - protein, 15 aa, Artificial Sequence
  Description of Artificial Sequence Synthetic peptide
DPDVMRYVDRYGQLQ

SEQ ID NO: 433 - protein, 15 aa, Artificial Sequence
  Description of Artificial Sequence Synthetic peptide
DSIAEVECFLTPEMG

SEQ ID NO: 434 - protein, 15 aa, Artificial Sequence
  Description of Artificial Sequence Synthetic peptide
DTLPCYSVARIPLPN

SEQ ID NO: 435 - protein, 15 aa, Artificial Sequence
  Description of Artificial Sequence Synthetic peptide
DVLPCYSVARIPLPN

SEQ ID NO: 436 - protein, 15 aa, Artificial Sequence
  Description of Artificial Sequence Synthetic peptide
EDLTCGNIPMWEAVT

SEQ ID NO: 437 - protein, 15 aa, Artificial Sequence
  Description of Artificial Sequence Synthetic peptide
EELPEDPDMMRYVDR

SEQ ID NO: 438 - protein, 15 aa, Artificial Sequence
  Description of Artificial Sequence Synthetic peptide
EELPGDPDMIRYVDR

SEQ ID NO: 439 - protein, 15 aa, Artificial Sequence
  Description of Artificial Sequence Synthetic peptide
LLDEFGVGPLCKGVN

SEQ ID NO: 440 - protein, 15 aa, Artificial Sequence
  Description of Artificial Sequence Synthetic peptide
LLTDLINRRTPKVDG

SEQ ID NO: 441 - protein, 15 aa, Artificial Sequence
  Description of Artificial Sequence Synthetic peptide
LLTDLINRRTPRIDG

SEQ ID NO: 442 - protein, 15 aa, Artificial Sequence
  Description of Artificial Sequence Synthetic peptide
LPILNEDLTCGNILM

SEQ ID NO: 443 - protein, 15 aa, Artificial Sequence
  Description of Artificial Sequence Synthetic peptide
MGDPDEHLRGFSKLI

SEQ ID NO: 444 - protein, 15 aa, Artificial Sequence
  Description of Artificial Sequence Synthetic peptide
MGDPNEHLRGFSKSI

SEQ ID NO: 445 - protein, 15 aa, Artificial Sequence
  Description of Artificial Sequence Synthetic peptide
MKMAPTKRKGERKDP

SEQ ID NO: 446 - protein, 15 aa, Artificial Sequence
  Description of Artificial Sequence Synthetic peptide
MMRYVDRYGQLQTKT

SEQ ID NO: 447 - protein, 15 aa, Artificial Sequence
  Description of Artificial Sequence Synthetic peptide
MMRYVDSCRQKCCNQ

SEQ ID NO: 448 - protein, 15 aa, Artificial Sequence
  Description of Artificial Sequence Synthetic peptide
MRYVDRYGQSQTMML

SEQ ID NO: 449 - protein, 15 aa, Artificial Sequence
  Description of Artificial Sequence Synthetic peptide
NRSGFQQWRGLSRYF

SEQ ID NO: 450 - protein, 15 aa, Artificial Sequence
  Description of Artificial Sequence Synthetic peptide
NRSGPQQWRGLSRYF

SEQ ID NO: 451 - protein, 15 aa, Artificial Sequence
  Description of Artificial Sequence Synthetic peptide
NVPPVLHITNTASTV

SEQ ID NO: 452 - protein, 15 aa, Artificial Sequence
  Description of Artificial Sequence Synthetic peptide
NVPPVLHITNTATTA

SEQ ID NO: 453 - protein, 15 aa, Artificial Sequence
  Description of Artificial Sequence Synthetic peptide
QPMYGMDAQVKEVRV

SEQ ID NO: 454 - protein, 15 aa, Artificial Sequence
  Description of Artificial Sequence Synthetic peptide
QSQVMNPEPKGYLDK

SEQ ID NO: 455 - protein, 15 aa, Artificial Sequence
  Description of Artificial Sequence Synthetic peptide
RKGRVKNPYPISFLL

SEQ ID NO: 456 - protein, 15 aa, Artificial Sequence
  Description of Artificial Sequence Synthetic peptide
RKRKVKNPYPISFLL

SEQ ID NO: 457 - protein, 15 aa, Artificial Sequence
  Description of Artificial Sequence Synthetic peptide
RKRRIKNPYPISFLL

SEQ ID NO: 458 - protein, 15 aa, Artificial Sequence
  Description of Artificial Sequence Synthetic peptide
RKRRVKDPYPISFLL

SEQ ID NO: 459 - protein, 15 aa, Artificial Sequence
  Description of Artificial Sequence Synthetic peptide
SKDMLPRFSVARIPL

SEQ ID NO: 460 - protein, 15 aa, Artificial Sequence
  Description of Artificial Sequence Synthetic peptide
SRYFKVELRKRRVKN

SEQ ID NO: 461 - protein, 15 aa, Artificial Sequence
  Description of Artificial Sequence Synthetic peptide
SRYFKVQLRKRKVKN

SEQ ID NO: 462 - protein, 15 aa, Artificial Sequence
  Description of Artificial Sequence Synthetic peptide
SRYFKVQLRKRRVKD

SEQ ID NO: 463 - protein, 15 aa, Artificial Sequence
  Description of Artificial Sequence Synthetic peptide
SRYFKVQPRKRRVKN

SEQ ID NO: 464 - protein, 15 aa, Artificial Sequence
  Description of Artificial Sequence Synthetic peptide
TEELPGDPDMITYVD

SEQ ID NO: 465 - protein, 15 aa, Artificial Sequence
  Description of Artificial Sequence Synthetic peptide
TIFPKNATVQSQVVN

SEQ ID NO: 466 - protein, 15 aa, Artificial Sequence
  Description of Artificial Sequence Synthetic peptide
TTGKLDEFGVGPLCK

SEQ ID NO: 467 - protein, 15 aa, Artificial Sequence
  Description of Artificial Sequence Synthetic peptide
TTVLLDDFGVGPLCK

SEQ ID NO: 468 - protein, 15 aa, Artificial Sequence
  Description of Artificial Sequence Synthetic peptide
TTVLLDEFGAGPLCK

SEQ ID NO: 469 - protein, 15 aa, Artificial Sequence
  Description of Artificial Sequence Synthetic peptide
TTVLLDEFGVRPLCK

SEQ ID NO: 470 - protein, 15 aa, Artificial Sequence
  Description of Artificial Sequence Synthetic peptide
TTVLLDELGVGPLCK

SEQ ID NO: 471 - protein, 15 aa, Artificial Sequence
  Description of Artificial Sequence Synthetic peptide
KASSTCKTPKRQCIP

SEQ ID NO: 472 - protein, 15 aa, Artificial Sequence
  Description of Artificial Sequence Synthetic peptide
KRWVKNPYPVVNLIN

SEQ ID NO: 473 - protein, 15 aa, Artificial Sequence
  Description of Artificial Sequence Synthetic peptide
LDENGVGPLCKGDGL

SEQ ID NO: 474 - protein, 15 aa, Artificial Sequence
  Description of Artificial Sequence Synthetic peptide
LDLQGLVLDYQTQYP

SEQ ID NO: 475 - protein, 15 aa, Artificial Sequence
  Description of Artificial Sequence Synthetic peptide
LRKRWVKNPYPVVNL

SEQ ID NO: 476 - protein, 15 aa, Artificial Sequence
  Description of Artificial Sequence Synthetic peptide
MFAIGEEPLDLQGLV

SEQ ID NO: 477 - protein, 15 aa, Artificial Sequence
  Description of Artificial Sequence Synthetic peptide
MFAIGGEPLDLQGLV

SEQ ID NO: 478 - protein, 15 aa, Artificial Sequence
  Description of Artificial Sequence Synthetic peptide
NEDITCDTLQMWEAI

SEQ ID NO: 479 - protein, 15 aa, Artificial Sequence
  Description of Artificial Sequence Synthetic peptide
NKDGNYPIEVWCPDP

SEQ ID NO: 480 - protein, 15 aa, Artificial Sequence
  Description of Artificial Sequence Synthetic peptide
PGDPDIVRFLDKFGQ

SEQ ID NO: 481 - protein, 15 aa, Artificial Sequence
  Description of Artificial Sequence Synthetic peptide
RVSLPMLNEDITCDT

SEQ ID NO: 482 - protein, 15 aa, Artificial Sequence
  Description of Artificial Sequence Synthetic peptide
SLINVHYWDMKRVHD

SEQ ID NO: 483 - protein, 15 aa, Artificial Sequence
  Description of Artificial Sequence Synthetic peptide
SPDLPTTSNWYTYTY

SEQ ID NO: 484 - protein, 15 aa, Artificial Sequence
  Description of Artificial Sequence Synthetic peptide
TTVLLDENGVGPLCK

SEQ ID NO: 485 - protein, 15 aa, Artificial Sequence
  Description of Artificial Sequence Synthetic peptide
VGISSLINVHYWVMK

SEQ ID NO: 486 - protein, 15 aa, Artificial Sequence
  Description of Artificial Sequence Synthetic peptide
VHDYGAGIPVSGVNY

SEQ ID NO: 487 - protein, 15 aa, Artificial Sequence
  Description of Artificial Sequence Synthetic peptide
VHYWVMKRVHDYGAG

SEQ ID NO: 488 - protein, 15 aa, Artificial Sequence
  Description of Artificial Sequence Synthetic peptide
YEGSEPLPGDPDIVR

SEQ ID NO: 489 - protein, 15 aa, Artificial Sequence
  Description of Artificial Sequence Synthetic peptide
APKKPKEPVQVPKLV

SEQ ID NO: 490 - protein, 15 aa, Artificial Sequence
  Description of Artificial Sequence Synthetic peptide
AVVGEPLELQGVLAN

SEQ ID NO: 491 - protein, 15 aa, Artificial Sequence
  Description of Artificial Sequence Synthetic peptide
KMAPAKRKGSCPGAA

SEQ ID NO: 492 - protein, 15 aa, Artificial Sequence
  Description of Artificial Sequence Synthetic peptide
MKMAPTKRKGSCPGA

SEQ ID NO: 493 - protein, 15 aa, Artificial Sequence
  Description of Artificial Sequence Synthetic peptide
TTVLLDEQGAGPLCK

SEQ ID NO: 494 - protein, 15 aa, Artificial Sequence
  Description of Artificial Sequence Synthetic peptide
GSHMGGVDVLAAVPL

SEQ ID NO: 495 - protein, 15 aa, Artificial Sequence
  Description of Artificial Sequence Synthetic peptide
KGGVDVLSAVPLSEE

SEQ ID NO: 496 - protein, 15 aa, Artificial Sequence
  Description of Artificial Sequence Synthetic peptide
MACTAKPACTPKPGR

SEQ ID NO: 497 - protein, 15 aa, Artificial Sequence
  Description of Artificial Sequence Synthetic peptide
NQVSECDMIIWELYR

SEQ ID NO: 498 - protein, 15 aa, Artificial Sequence
  Description of Artificial Sequence Synthetic peptide
PDIPNQVSECDMIIW

SEQ ID NO: 499 - protein, 15 aa, Artificial Sequence
  Description of Artificial Sequence Synthetic peptide
AITQIEAYLNPRMGN

SEQ ID NO: 500 - protein, 15 aa, Artificial Sequence
  Description of Artificial Sequence Synthetic peptide
ATTPPVMQFTNSVTT

SEQ ID NO: 501 - protein, 15 aa, Artificial Sequence
  Description of Artificial Sequence Synthetic peptide
DIVGIHTNYSESQNW

SEQ ID NO: 502 - protein, 15 aa, Artificial Sequence
  Description of Artificial Sequence Synthetic peptide
EGLPGDPDLDRYVDK

SEQ ID NO: 503 - protein, 15 aa, Artificial Sequence
  Description of Artificial Sequence Synthetic peptide
FTGGATTPPVMQFTN

SEQ ID NO: 504 - protein, 15 aa, Artificial Sequence
  Description of Artificial Sequence Synthetic peptide
IEAYLNPRMGNNNPT

SEQ ID NO: 505 - protein, 15 aa, Artificial Sequence
  Description of Artificial Sequence Synthetic peptide
KTCPTPAPVPKLLVK

SEQ ID NO: 506 - protein, 15 aa, Artificial Sequence
  Description of Artificial Sequence Synthetic peptide
LNPRMGNNNPTDELY

SEQ ID NO: 507 - protein, 15 aa, Artificial Sequence
  Description of Artificial Sequence Synthetic peptide
MWEAVSVKTEVMGIS

SEQ ID NO: 508 - protein, 15 aa, Artificial Sequence
  Description of Artificial Sequence Synthetic peptide
GNPTLSDAYSQQRSV

SEQ ID NO: 509 - protein, 15 aa, Artificial Sequence
  Description of Artificial Sequence Synthetic peptide
MLGMVGYAGNPTLSD

SEQ ID NO: 510 - protein, 15 aa, Artificial Sequence
  Description of Artificial Sequence Synthetic peptide
NQSTTPLVDENGVGI

SEQ ID NO: 511 - protein, 15 aa, Artificial Sequence
  Description of Artificial Sequence Synthetic peptide
QKPQVQGTQPNAVQE

SEQ ID NO: 512 - protein, 15 aa, Artificial Sequence
  Description of Artificial Sequence Synthetic peptide
TAVYQSRGAPYTFTD

SEQ ID NO: 513 - protein, 15 aa, Artificial Sequence
  Description of Artificial Sequence Synthetic peptide
TRKQVTAANFPIEIW

SEQ ID NO: 514 - protein, 15 aa, Artificial Sequence
  Description of Artificial Sequence Synthetic peptide
VTAANFPIEIWSADP

SEQ ID NO: 515 - protein, 15 aa, Artificial Sequence
  Description of Artificial Sequence Synthetic peptide
YKVEAILLPNFASGS

SEQ ID NO: 516 - protein, 15 aa, Artificial Sequence
  Description of Artificial Sequence Synthetic peptide
AKISVAPKKNTDKKE

SEQ ID NO: 517 - protein, 15 aa, Artificial Sequence
  Description of Artificial Sequence Synthetic peptide
APTSKFLLQNGELIY

SEQ ID NO: 518 - protein, 15 aa, Artificial Sequence
  Description of Artificial Sequence Synthetic peptide
DAMCEDTMIVWEAYR

SEQ ID NO: 519 - protein, 15 aa, Artificial Sequence
  Description of Artificial Sequence Synthetic peptide
EDTMIVWEAYRLETE

SEQ ID NO: 520 - protein, 15 aa, Artificial Sequence
  Description of Artificial Sequence Synthetic peptide
FFRVHCRQRRIKHPY

SEQ ID NO: 521 - protein, 15 aa, Artificial Sequence
  Description of Artificial Sequence Synthetic peptide
GPLDVIGINPDPERL

SEQ ID NO: 522 - protein, 15 aa, Artificial Sequence
  Description of Artificial Sequence Synthetic peptide
ISVAPKKNTDNKKEL

SEQ ID NO: 523 - protein, 15 aa, Artificial Sequence
  Description of Artificial Sequence Synthetic peptide
RKQVNAANFPVELWV

SEQ ID NO: 524 - protein, 15 aa, Artificial Sequence
  Description of Artificial Sequence Synthetic peptide
WACGGGPLDVIGINP

SEQ ID NO: 525 - protein, 15 aa, Artificial Sequence
  Description of Artificial Sequence Synthetic peptide
ANQRTAPQWMLPLLL

SEQ ID NO: 526 - protein, 15 aa, Artificial Sequence
  Description of Artificial Sequence Synthetic peptide
EYYSDLSPIRPSMVR

SEQ ID NO: 527 - protein, 15 aa, Artificial Sequence
  Description of Artificial Sequence Synthetic peptide
MALELFNPDEYYDIL

SEQ ID NO: 528 - protein, 15 aa, Artificial Sequence
  Description of Artificial Sequence Synthetic peptide
WHVIRDDIPAITSQE

SEQ ID NO: 529 - protein, 15 aa, Artificial Sequence
  Description of Artificial Sequence Synthetic peptide
APGGANQRSAPQWML

SEQ ID NO: 530 - protein, 15 aa, Artificial Sequence
  Description of Artificial Sequence Synthetic peptide
EDYYDILFPGVNAFV

SEQ ID NO: 531 - protein, 15 aa, Artificial Sequence
  Description of Artificial Sequence Synthetic peptide
KVSTVGLFQQPAMAL

SEQ ID NO: 532 - protein, 15 aa, Artificial Sequence
  Description of Artificial Sequence Synthetic peptide
MALQLFNPEDYYDIL

SEQ ID NO: 533 - protein, 15 aa, Artificial Sequence
  Description of Artificial Sequence Synthetic peptide
PGVNAFVNNIHYLDP

SEQ ID NO: 534 - protein, 15 aa, Artificial Sequence
  Description of Artificial Sequence Synthetic peptide
YLDPRHWGPSLFSTI

SEQ ID NO: 535 - protein, 15 aa, Artificial Sequence
  Description of Artificial Sequence Synthetic peptide
YYSRLSPVRPSMVRQ

SEQ ID NO: 536 - protein, 15 aa, Artificial Sequence
  Description of Artificial Sequence Synthetic peptide
FNALSEGVHRLGQWI

SEQ ID NO: 537 - protein, 15 aa, Artificial Sequence
  Description of Artificial Sequence Synthetic peptide
RERELLQIAAGQPVD

SEQ ID NO: 538 - protein, 15 aa, Artificial Sequence
  Description of Artificial Sequence Synthetic peptide
RIAYGIWTSYYNTGR

SEQ ID NO: 539 - protein, 15 aa, Artificial Sequence
  Description of Artificial Sequence Synthetic peptide
VVNRAVSEELQRLLG

SEQ ID NO: 540 - protein, 15 aa, Artificial Sequence
  Description of Artificial Sequence Synthetic peptide
ASLATVEGITTTSEA

SEQ ID NO: 541 - protein, 15 aa, Artificial Sequence
  Description of Artificial Sequence Synthetic peptide
DYYSNLSPIRPSMVR

SEQ ID NO: 542 - protein, 15 aa, Artificial Sequence
  Description of Artificial Sequence Synthetic peptide
IAGFAALIQTVTGVS

SEQ ID NO: 543 - protein, 15 aa, Artificial Sequence
  Description of Artificial Sequence Synthetic peptide
LLGLYGTVTPALAAY

SEQ ID NO: 544 - protein, 15 aa, Artificial Sequence
  Description of Artificial Sequence Synthetic peptide
TVGVRLSREQVSLVN

SEQ ID NO: 545 - protein, 15 aa, Artificial Sequence
  Description of Artificial Sequence Synthetic peptide
AIDQYMVVFEDVKGT

SEQ ID NO: 546 - protein, 15 aa, Artificial Sequence
  Description of Artificial Sequence Synthetic peptide
CLLPKMDSVIFDFLH

SEQ ID NO: 547 - protein, 15 aa, Artificial Sequence
  Description of Artificial Sequence Synthetic peptide
DFATDIQSRIVEWKE

SEQ ID NO: 548 - protein, 15 aa, Artificial Sequence
  Description of Artificial Sequence Synthetic peptide
DIQSRIVEWKERLDS

SEQ ID NO: 549 - protein, 15 aa, Artificial Sequence
  Description of Artificial Sequence Synthetic peptide
EELHLCKGFQCFKRP

SEQ ID NO: 550 - protein, 15 aa, Artificial Sequence
  Description of Artificial Sequence Synthetic peptide
ELGVAIDQYMVVFED

SEQ ID NO: 551 - protein, 15 aa, Artificial Sequence
  Description of Artificial Sequence Synthetic peptide
ESMELMDLLGLERAA

SEQ ID NO: 552 - protein, 15 aa, Artificial Sequence
  Description of Artificial Sequence Synthetic peptide
EYLLYSALTRDPYHT

SEQ ID NO: 553 - protein, 15 aa, Artificial Sequence
  Description of Artificial Sequence Synthetic peptide
FFLTPHRHRVSAINN

SEQ ID NO: 554 - protein, 15 aa, Artificial Sequence
  Description of Artificial Sequence Synthetic peptide
FLHCIVFNVPKRRYW

SEQ ID NO: 555 - protein, 15 aa, Artificial Sequence
  Description of Artificial Sequence Synthetic peptide
GGDEDKMKRMNTLYK

SEQ ID NO: 556 - protein, 15 aa, Artificial Sequence
  Description of Artificial Sequence Synthetic peptide
HGINNLDSLRDYLDG

SEQ ID NO: 557 - protein, 15 aa, Artificial Sequence
  Description of Artificial Sequence Synthetic peptide
KRVDTLHMTREEMLT

SEQ ID NO: 558 - protein, 15 aa, Artificial Sequence
  Description of Artificial Sequence Synthetic peptide
KYSVTFISRHMCAGH

SEQ ID NO: 559 - protein, 15 aa, Artificial Sequence
  Description of Artificial Sequence Synthetic peptide
LCKGFQCFKRPKTPP

SEQ ID NO: 560 - protein, 15 aa, Artificial Sequence
  Description of Artificial Sequence Synthetic peptide
LGLERAAWGNLPLMR

SEQ ID NO: 561 - protein, 15 aa, Artificial Sequence
  Description of Artificial Sequence Synthetic peptide
LMRKAYLRKCKEFHP

SEQ ID NO: 562 - protein, 15 aa, Artificial Sequence
  Description of Artificial Sequence Synthetic peptide
LNREESMELMDLLGL

SEQ ID NO: 563 - protein, 15 aa, Artificial Sequence
  Description of Artificial Sequence Synthetic peptide
MAGVAWLHCLLPKMD

SEQ ID NO: 564 - protein, 15 aa, Artificial Sequence
  Description of Artificial Sequence Synthetic peptide
MDKVLNREESMELMD

SEQ ID NO: 565 - protein, 15 aa, Artificial Sequence
  Description of Artificial Sequence Synthetic peptide
RVSAINNFCQKLCTF

SEQ ID NO: 566 - protein, 15 aa, Artificial Sequence
  Description of Artificial Sequence Synthetic peptide
TFSRMKYNICMGKCI

SEQ ID NO: 567 - protein, 15 aa, Artificial Sequence
  Description of Artificial Sequence Synthetic peptide
VKVNLEKKHLNKRTQ

SEQ ID NO: 568 - protein, 15 aa, Artificial Sequence
  Description of Artificial Sequence Synthetic peptide
LNINIPSEKLPFELG

SEQ ID NO: 569 - protein, 15 aa, Artificial Sequence
  Description of Artificial Sequence Synthetic peptide
LQKYQCSFISKHAFY

SEQ ID NO: 570 - protein, 15 aa, Artificial Sequence
  Description of Artificial Sequence Synthetic peptide
NKRSQIFPPGIVTMN

SEQ ID NO: 571 - protein, 15 aa, Artificial Sequence
  Description of Artificial Sequence Synthetic peptide
NNLDNLRDYLDGCVE

SEQ ID NO: 572 - protein, 15 aa, Artificial Sequence
  Description of Artificial Sequence Synthetic peptide
NVVYWKEVLDNYIGL

SEQ ID NO: 573 - protein, 15 aa, Artificial Sequence
  Description of Artificial Sequence Synthetic peptide
PGIVTMNEYCIPETV

SEQ ID NO: 574 - protein, 15 aa, Artificial Sequence
  Description of Artificial Sequence Synthetic peptide
PHKHRVSAINNFCKG

SEQ ID NO: 575 - protein, 15 aa, Artificial Sequence
  Description of Artificial Sequence Synthetic peptide
QCSFISKHAFYNTVL

SEQ ID NO: 576 - protein, 15 aa, Artificial Sequence
  Description of Artificial Sequence Synthetic peptide
TMNEYCIPETVAVRF

SEQ ID NO: 577 - protein, 15 aa, Artificial Sequence
  Description of Artificial Sequence Synthetic peptide
VHDLNEEEDNIWQSS

SEQ ID NO: 578 - protein, 15 aa, Artificial Sequence
  Description of Artificial Sequence Synthetic peptide
YKKLLQKYQCSFISK

SEQ ID NO: 579 - protein, 15 aa, Artificial Sequence
  Description of Artificial Sequence Synthetic peptide
YMASIAWYTGLNKKI

SEQ ID NO: 580 - protein, 15 aa, Artificial Sequence
  Description of Artificial Sequence Synthetic peptide
AIELYDKIEKFKVDF

SEQ ID NO: 581 - protein, 15 aa, Artificial Sequence
  Description of Artificial Sequence Synthetic peptide
AIYTTSDKAIELYDK

SEQ ID NO: 582 - protein, 15 aa, Artificial Sequence
  Description of Artificial Sequence Synthetic peptide
AVSLEKKHVNKKHQI

SEQ ID NO: 583 - protein, 15 aa, Artificial Sequence
  Description of Artificial Sequence Synthetic peptide
CKKFKKHLERLRDLD

SEQ ID NO: 584 - protein, 15 aa, Artificial Sequence
  Description of Artificial Sequence Synthetic peptide
CLIWCLPDTTFKPCL

SEQ ID NO: 585 - protein, 15 aa, Artificial Sequence
  Description of Artificial Sequence Synthetic peptide
CLPDTTFKPCLQEEI

SEQ ID NO: 586 - protein, 15 aa, Artificial Sequence
  Description of Artificial Sequence Synthetic peptide
DLDTIDLLYYMGGVA

SEQ ID NO: 587 - protein, 15 aa, Artificial Sequence
  Description of Artificial Sequence Synthetic peptide
EKKLQKIIQLLTENI

SEQ ID NO: 588 - protein, 15 aa, Artificial Sequence
  Description of Artificial Sequence Synthetic peptide
ENIPKYRNIWFKGPI

SEQ ID NO: 589 - protein, 15 aa, Artificial Sequence
  Description of Artificial Sequence Synthetic peptide
ERLRDLDTIDLLYYM

SEQ ID NO: 590 - protein, 15 aa, Artificial Sequence
  Description of Artificial Sequence Synthetic peptide
HSQSSSSGYGSFSAS

SEQ ID NO: 591 - protein, 15 aa, Artificial Sequence
  Description of Artificial Sequence Synthetic peptide
KPFPCQKCENRSRLK

SEQ ID NO: 592 - protein, 15 aa, Artificial Sequence
  Description of Artificial Sequence Synthetic peptide
LCKLLEIAPNCYGNI

SEQ ID NO: 593 - protein, 15 aa, Artificial Sequence
  Description of Artificial Sequence Synthetic peptide
LEIAPNCYGNIPLMK

SEQ ID NO: 594 - protein, 15 aa, Artificial Sequence
  Description of Artificial Sequence Synthetic peptide
MDLVLNRKEREALCK

SEQ ID NO: 595 - protein, 15 aa, Artificial Sequence
  Description of Artificial Sequence Synthetic peptide
NKDLQPGQGINNLDN

SEQ ID NO: 596 - protein, 15 aa, Artificial Sequence
  Description of Artificial Sequence Synthetic peptide
NSSRTDGTWEDLFCD

SEQ ID NO: 597 - protein, 15 aa, Artificial Sequence
  Description of Artificial Sequence Synthetic peptide
PCLQEEIKNWKQILQ

SEQ ID NO: 598 - protein, 15 aa, Artificial Sequence
  Description of Artificial Sequence Synthetic peptide
QLLTENIPKYRNIWF

SEQ ID NO: 599 - protein, 15 aa, Artificial Sequence
  Description of Artificial Sequence Synthetic peptide
SVPRNSSRTDGTWED

SEQ ID NO: 600 - protein, 15 aa, Artificial Sequence
  Description of Artificial Sequence Synthetic peptide
TDGTWEDLFCDESLS

SEQ ID NO: 601 - protein, 15 aa, Artificial Sequence
  Description of Artificial Sequence Synthetic peptide
TPVPTDFPIDLSDYL

SEQ ID NO: 602 - protein, 15 aa, Artificial Sequence
  Description of Artificial Sequence Synthetic peptide
EDLLARRFEKILDKM

SEQ ID NO: 603 - protein, 15 aa, Artificial Sequence
  Description of Artificial Sequence Synthetic peptide
EEKMKKLNSLYLKLQ

SEQ ID NO: 604 - protein, 15 aa, Artificial Sequence
  Description of Artificial Sequence Synthetic peptide
EFVSQAVFSNRTLTA

SEQ ID NO: 605 - protein, 15 aa, Artificial Sequence
  Description of Artificial Sequence Synthetic peptide
EKILDKMDKTIKGEQ

SEQ ID NO: 606 - protein, 15 aa, Artificial Sequence
  Description of Artificial Sequence Synthetic peptide
ELGVAIDQFTVVFED

SEQ ID NO: 607 - protein, 15 aa, Artificial Sequence
  Description of Artificial Sequence Synthetic peptide
ESLDKTPELMVKRVL

SEQ ID NO: 608 - protein, 15 aa, Artificial Sequence
  Description of Artificial Sequence Synthetic peptide
FILTPFRHRVSAVNN

SEQ ID NO: 609 - protein, 15 aa, Artificial Sequence
  Description of Artificial Sequence Synthetic peptide
GEFKDQLNWKALSEF

SEQ ID NO: 610 - protein, 15 aa, Artificial Sequence
  Description of Artificial Sequence Synthetic peptide
GEQDVLLYMAGVAWY

SEQ ID NO: 611 - protein, 15 aa, Artificial Sequence
  Description of Artificial Sequence Synthetic peptide
GLNGKIDELVYRYLK

SEQ ID NO: 612 - protein, 15 aa, Artificial Sequence
  Description of Artificial Sequence Synthetic peptide
GNGMSNLDNLRDYLD

SEQ ID NO: 613 - protein, 15 aa, Artificial Sequence
  Description of Artificial Sequence Synthetic peptide
GNLPLMRTKYLSKCK

SEQ ID NO: 614 - protein, 15 aa, Artificial Sequence
  Description of Artificial Sequence Synthetic peptide
KCNFASRHSYYNTAL

SEQ ID NO: 615 - protein, 15 aa, Artificial Sequence
  Description of Artificial Sequence Synthetic peptide
KDNATDASLSFPKEL

SEQ ID NO: 616 - protein, 15 aa, Artificial Sequence
  Description of Artificial Sequence Synthetic peptide
LDKYIGLTEFADMQM

SEQ ID NO: 617 - protein, 15 aa, Artificial Sequence
  Description of Artificial Sequence Synthetic peptide
LEKKHLNKRSQIFPP

SEQ ID NO: 618 - protein, 15 aa, Artificial Sequence
  Description of Artificial Sequence Synthetic peptide
LKENDFKAEDLYGEF

SEQ ID NO: 619 - protein, 15 aa, Artificial Sequence
  Description of Artificial Sequence Synthetic peptide
NAYGLYSRMTRDPFT

SEQ ID NO: 620 - protein, 15 aa, Artificial Sequence
  Description of Artificial Sequence Synthetic peptide
PGIVTMNEYLVPATL

SEQ ID NO: 621 - protein, 15 aa, Artificial Sequence
  Description of Artificial Sequence Synthetic peptide
PKKKKDNATDASLSF

SEQ ID NO: 622 - protein, 15 aa, Artificial Sequence
  Description of Artificial Sequence Synthetic peptide
SSSQIPTYGTPDWDY

SEQ ID NO: 623 - protein, 15 aa, Artificial Sequence
  Description of Artificial Sequence Synthetic peptide
VIHTTKEKAETLYKK

SEQ ID NO: 624 - protein, 15 aa, Artificial Sequence
  Description of Artificial Sequence Synthetic peptide
VKVNLEKKHLNKRSQ

SEQ ID NO: 625 - protein, 15 aa, Artificial Sequence
  Description of Artificial Sequence Synthetic peptide
CGGKSLNVNMPLEKL

SEQ ID NO: 626 - protein, 15 aa, Artificial Sequence
  Description of Artificial Sequence Synthetic peptide
DFAQDIQSRIVEWKE

SEQ ID NO: 627 - protein, 15 aa, Artificial Sequence
  Description of Artificial Sequence Synthetic peptide
DSGHGSSTESQSQCC

SEQ ID NO: 628 - protein, 15 aa, Artificial Sequence
  Description of Artificial Sequence Synthetic peptide
EYLLYSALTREPYHT

SEQ ID NO: 629 - protein, 15 aa, Artificial Sequence
  Description of Artificial Sequence Synthetic peptide
GSVRVNLERKHQNKR

SEQ ID NO: 630 - protein, 15 aa, Artificial Sequence
  Description of Artificial Sequence Synthetic peptide
WQKTLEETDYCLLHL

SEQ ID NO: 631 - protein, 15 aa, Artificial Sequence
  Description of Artificial Sequence Synthetic peptide
HQPDFGGFWDATEVF

SEQ ID NO: 632 - protein, 15 aa, Artificial Sequence
  Description of Artificial Sequence Synthetic peptide
LRMKHENRKLYRKDP

SEQ ID NO: 633 - protein, 15 aa, Artificial Sequence
  Description of Artificial Sequence Synthetic peptide
PGVDAIYCKQWPECA

SEQ ID NO: 634 - protein, 15 aa, Artificial Sequence
  Description of Artificial Sequence Synthetic peptide
FYYLCNCFYCFLDKR

SEQ ID NO: 635 - protein, 15 aa, Artificial Sequence
  Description of Artificial Sequence Synthetic peptide
KIFRKPPMWIECYCY

SEQ ID NO: 636 - protein, 15 aa, Artificial Sequence
  Description of Artificial Sequence Synthetic peptide
YCYRCYREWFGFEIS

SEQ ID NO: 637 - protein, 15 aa, Artificial Sequence
  Description of Artificial Sequence Synthetic peptide
CADFPLCPDTLYCKD

SEQ ID NO: 638 - protein, 15 aa, Artificial Sequence
  Description of Artificial Sequence Synthetic peptide
EKMKKMNTLYKKMEQ

SEQ ID NO: 639 - protein, 15 aa, Artificial Sequence
  Description of Artificial Sequence Synthetic peptide
GNLSLMRKAYLRKCK

SEQ ID NO: 640 - protein, 15 aa, Artificial Sequence
  Description of Artificial Sequence Synthetic peptide
LGLERAAWGNLSLMR

SEQ ID NO: 641 - protein, 15 aa, Artificial Sequence
  Description of Artificial Sequence Synthetic peptide
RAAWGNLSLMRKAYL

SEQ ID NO: 642 - protein, 15 aa, Artificial Sequence
  Description of Artificial Sequence Synthetic peptide
LLEFCRGEDSVDGKN

SEQ ID NO: 643 - protein, 15 aa, Artificial Sequence
  Description of Artificial Sequence Synthetic peptide
QASVKVSKTWTGTKK

SEQ ID NO: 644 - protein, 15 aa, Artificial Sequence
  Description of Artificial Sequence Synthetic peptide
KVRRSWTESKKTAQR

SEQ ID NO: 645 - protein, 15 aa, Artificial Sequence
  Description of Artificial Sequence Synthetic peptide
LLEFCRGKDSVDGKN

SEQ ID NO: 646 - protein, 15 aa, Artificial Sequence
  Description of Artificial Sequence Synthetic peptide
MVLRQLSRQASVKIG

SEQ ID NO: 647 - protein, 15 aa, Artificial Sequence
  Description of Artificial Sequence Synthetic peptide
QASVKIGKTWTGTKK

(19) United States
(12) Patent Application Publication
(10) Pub. No.: US 2015/0065367 A1
(43) Pub. Date: Mar. 5, 2015

(54) POLYOMAVIRUS PEPTIDE SEQUENCES

(71) Applicant: Janssen Diagnostics BVBA, Beerse (BE)

(72) Inventor: Lieven Jozef Stuyver, Herzele (BE)

(21) Appl. No.: 14/364,776

(22) PCT Filed: Dec. 11, 2012
(86) PCT No.: PCT/EP2012/075031

§ 371 Date: Jun. 12, 2014

(30) Foreign Application Priority Data
Dec. 12, 2011 (EP) 11193083.0
Dec. 11, 2012 (EP) 12798747.7

(51) Int. Cl. G01N 33/569

(52) U.S. Cl. CPC G01N 33/56983; G01N 2333/025
USPC 506/9; 530/326

(57) ABSTRACT

The current invention concerns the identification of B-cell epitopes (as linear peptides) from human polyoma virus proteins and their use in an immune diagnostic assay.

The current invention relates to the identification of B-cell epitopes (as linear peptides) from human polyoma virus proteins and their use in an immune diagnostic assay.

Progressive multifocal leukoencephalopathy (PML) is a rare but often fatal brain disease caused by reactivation of the polyomavirus JC. The monoclonal antibodies natalizumab, efalizumab, and rituximab—used for the treatment of multiple sclerosis, psoriasis, hematological malignancies, Crohn's disease, and rheumatic diseases—have been associated with PML. Worldwide 181 (as of November 2011) cases of natalizumab-associated PML have been reported. International studies and standardization of methods are urgently needed to devise strategies to mitigate the risk of PML in natalizumab-treated patients.

A new set of assay developments could lead to a better understanding of the virus reactivation, and that could lead to safe use of immune modulating agents (e.g. a Tysabri® (natalizumab)) and an optimized treatment algorithm.

### BACKGROUND

The human neurotropic polyomavirus JCV is a non-enveloped DNA virus belonging to the group of polyomaviruses. JCV is the etiologic agent of progressive multifocal leukoencephalopathy (PML). Other members of this viral family are BK virus (mainly infecting the kidneys), and the non-human SV40 virus. JC and BK viruses have been named using the initials of the first patients discovered with the diseases.

Epidemiological studies showed that in certain populations, the seroprevalence of close to 90% by age 20. In those healthy immunocompetent individuals, JCV is establishing a lifelong sub-clinical infection.

The initial site of infection may be the tonsils, or possibly the gastrointestiinal tract. The virus remains latent and/or can infect the tubular epithelial cells in the kidneys where it continues to reproduce, thereby shedding virus particles in the urine. JCV can cross the blood-brain barrier, and enters into the central nervous system where it infects oligodendrocytes and astrocytes.

Immunodeficiency or immuno-suppression allows JCV to reactivate. In the brain, this will cause the usually fatal PML by destroying oligodendrocytes.

Therefore, PML is a demyleating disease affecting the white matter, but is in process different from multiple sclerosis (MS), in which the myelin itself is destroyed. Whether the process behind PML is caused by the reactivation of JCV within the CNS or seeding of newly reactivated JCV via blood or lymphatics is unknown. PML progresses much more quickly than MS.

There are case reports of PML being induced by pharmacological agents (efalizumab, rituximab, infliximab, natalizumab . . . ) but the process how JCV interacts with these mAbs and cause PML is again not clearly understood.

PML is diagnosed by testing for JC virus DNA in cerebrosinal fluid, or in brain biopsy specimens. In addition, brain damage caused by PML has been detected on MRI images.

As of today, there is no known cure for PML, but the disease can be slowed or stopped, dependent on improvement of the patient's immune restoration (e.g. HAART in AIDS patients). A rare complication of immune reconstitution is known as “immune reconstitution inflammatory syndrom (IRIS), in which increased immune system activity increases the damage caused by the infection. IRIS can be managed by pharmacological intervention, but it is extremely fatal if it occurs in PML.

### Access to Clinical Isolates

In order to study the correlates of JCV and PML, a large collection of clinical samples is needed, inclusive with the individual's clinical background.

JCV replicates in several different types of tissues (tonsils, gastro-intestinal tract, kidney, brain). In order to obtain a representative set of genetic variants and the corresponding serological markers, it is aimed to start with the collection of a large sample set from urine, blood, CSF, bone marrow, and paraffin embedded brain biopsy material, and potentially tonsil biopsy. Blood cells can be separated into different compartments (FACS). PML is a rare disease present only in immune suppressed individuals, and access to these precious materials is foreseen to be limited. Most of the study objectives for assay design can be completed on samples from infected healthy individuals.

### The Genetic Variability of JCV (Aenotvoes and Variants) and Tropism

Sequencing of the JCV genome indicates at least seven major genotypes and numerous subtypes. The type distribution was found to be as follows: Type 1: in Europeans; Types 2 and 7: in Asians; Types 3 and 6: in Africans; Type 4: in the United States, the whole genome of Type 4 strains was found to be most closely related to Type 1; and Type 5: a single natural occurring recombinant strain of Type 6 in VP1 gene with Type 2B in the early region. These genotypes and subtypes have been defined in three ways: namely by i) a 610 bp region spanning the 3′ ends of the VP1 and T-antigen genes, ii) a 215 bp region of the 5′ end of the VP1 gene and iii) based on the sequence of the entire coding region of the genome (5130 bp in strain MAD-1; Accession number: PLYCG MAD-1) including untranslated regions except the archetypal regulatory region to the late side of on.

Besides the genotypic variations, the regulatory domain and the VP1 region contains mutations that are found more frequently in PML patients. From the frequency of observation, it is thought that these mutations are positively selected, and are not just present by chance. Analysis of the VP1 sequences isolated from PML patients were compared to control samples from healthy individuals showing that the mutated residues are located within the sialic acid binding site, a JC virus receptor for cell infection. It is therefore likely that a more virulent PML-causing phenotype of JC virus is acquired via adaptive evolution that changes viral specificity for its cellular receptor(s).

On the other hand, on the basis of the survival time (less or more than 6 months) from the onset of the disease, patients were grouped in slow and fast PML progressors (SP and FP PML). It was suggested that VP1 outer loops can contain polymorphic residues restricted to four positions (aa 74, 75, 117 and 128) in patients with slow PML progression, VP1 loop mutations are associated with a favorable prognosis for PML.

The genomic organization and variability of JCV in the transcriptional control region (TCR), a 400 base pare non-coding regulatory region, were described by Jensen (2001). In addition, distinctive point mutations or deletions in the regulatory region also provide useful information to supplement coding region typing.

Rearranged JCV regulatory regions (RR), including tandem repeat patterns found in the central nervous system (CNS) of PML patients, have been associated with neurovirulence.

In HIV-infected patients with virologically confirmed PML, highly active antiretroviral therapy (HAART) leads to a partial immune-mediated control of JCV replication in CSF. Hoverer, the virus may tend to escape through the selection of rearrangements in the RR, some associated with enhanced viral replication efficiency, other resulting in multiplication of binding sites for cellular transcription factors (Macrophage Chemoattractant Protein MCP-1; cellular transcription factor NF-1). In a case of PML in an HIV-1 infected individual that did not respond to HAART therapy, there was a simultaneous presence of JCV strains with four different TCR structures in urine, peripheral blood cells, serum, and CSF samples, for which the authors suggested that the archetype TCR is restricted to urine, while the degree of the rearrangement varies and increases from the peripheral blood to CSF.

It is currently not clear if PML is more frequently found within certain genotypes, or if certain genotypes are excluded from PML. Also the genetic polymorphisms in VP1 and the RR need further analysis in the context of the different genotypes, tissue distribution, and presence/absence of PML. While infection is very common in most human populations, this is usually subclinical since the virus is readily controlled by the immune system. After the initial infection is resolved. JCV nonetheless persists in the body and enters a state of latency which is poorly understood. However, under circumstances in which the immune system becomes impaired, e.g., AIDS, the virus reactivates and replicates in the central nervous system (CNS) to cause PML. The mechanisms involved in this reactivation are not known but it is possible that changes in the levels of cytokines and immunomodulators, such as TNF-α, MIP-1α and TGF-β, that are associated with immunosuppression, elicit changes in intracellular signal transduction pathways that, in turn, modulate the activities of transcription factors (e.g. Sp1 and Egr-1) that bound to the GG(A/C)-rich sequences in the TCR. These transcription factors are involved in regulating the expression of JCV genes.

JCV DNA is frequently, but intermittently detected in peripheral blood, supporting the hypothesis of viral reservoirs. In addition, mRNAs were seldom associated with DNA, suggesting that JCV reactivation does not take place in peripheral blood. JCV might remain latent in the peripheral reservoir, and immune suppression might enable reactivation, thereby facilitating the detection of JCV DNA in blood. However, circulating virus might have no link to the emergence of PML.

### JCV Natural History

Antibody titers to JCV were measured in the past with hemagglutination inhibition (HI) assays. Nowadays, hemagglutination- and HI-assays are only used to study modifications in Vp1, and the effect of these mutations on receptor recognition. HI assays are replaced by antibody detection technologies. The detected antibodies to JCV are against Vp1 epitopes, the protein that makes up 75% of the total virion protein.

Recently, in addition to the previously characterized viruses BK and JC, three new human polyomaviruses have been identified: KIV (respiratory tract infection), WUV (respiratory tract infection), and MCV (merkel cell carcinoma). It was determined that initial exposure to KIV, WUV, and MCV occurs in childhood, similar to that for the known human polyomaviruses BKV and JCV, and that their prevalence is high. In order to study exposure to these viruses in humans, recombinant polyomavirus VP1 capsid proteins were expressed in E. coli in an ELISA assay.

Sera of 1501 adult individuals were tested for the presence of 7 polyomaviruses (including SV40=primate virus, in humans through the SV40-contaminated polio vaccine: and LPV=lymphotropic polyoma virus in African green monkeys) and the authors indicated that there may be an age-related waning of BKV VP1 specific antibodies, but not for the other 6 polyomaviruses tested. Also, a difference in sero-prevalence with respect to gender for any of the 7 polyomaviruses tested was not found (Kean et al., 2009). Of the 195 samples exhibiting initial SV40 seroreactivity, only 7 (3%) were cross reactive with JCV Vp1 protein. No other cross reactivity with JCV Vp1 was observed.

Since there is a causal relationship of reactivation of JCV in CSF and the development of PML, knowing the JCV serological status of individuals with decreased immunological status is crucial. Theoretically, uninfected individuals (seronegative) should not be at risk for developing PML, while seropositive individuals are. There are case reports of PML being caused by pharmacological agents, although there is some speculation this could be due in part to the existing impaired immune response or ‘drug combination therapies’ rather than individual drugs. These include efalizumab, rituximab, belatacept, infliximab, natalizumab, chemotherapy, corticosteroids, and various transplant drugs such as tacrolimus.

Epidemiological studies suggest that the JCV infection occurs primarily in childhood, but the infection in adults is not excluded. Seronegative individuals undergoing immunesuppression and/or therapy should in generally not at risk, but they might be in the seroconversion window where antibodies are not yet properly available. Hence this population would require further attention and analysis by molecular diagnostic means. The sensitivity and specificity of a JC virus serology assay is of substantial interest because such an assay is now being considered as a means to assess the risk of PML in patients treated with natalizumab.

Current available immune-assays are based on VP1 only, expressed in a baculovirus expression system, in an E. coli expression system or in a yeast expression system. No other viral proteins are available in such an assay meaning that only so-called conformational epitopes, but not linear epitopes present in the three dimensional structure of the virus, are part of the immune assay. As a consequence thereof human samples potentially containing antibodies directed against the missing part as such, will not be detected.

As a final Tysabri treatment algorithm would require the knowledge of the infection status, there is a high unmet medical need to:
design serological assays for JCV anti-IgG and anti-IgM, and confirm the serological specificity of the JCV assay against other polyomaviruses.
compare the serological assay results to a ‘gold standard’ molecular assay with detection limit of ˜50 viral copies/ml generating information on sensitivity, specificity, positive and negative predictive values.
convert the serological assay to a point of care technology.
explore the serological status in a large collection of healthy individuals and in different groups of patients.
compare the serology assay with the cellular immune response assay.

The current invention therefore relates to human polyoma virus peptide sequences possessing an immune activity towards human antibodies in human samples.

More specifically the current invention makes it unexpectedly possible to use the human polyoma viral small T antigen for immune response diagnostic purposes.

The 63 specific sequences identified in Table 9 are considered human polyoma viral immune-dominant epitopes as indicated for the several polyoma viruses and can be used for immune diagnostic purposes accordingly.

In addition the human polyoma virus peptide sequences can be used for B-cell epitope studies i.e. the identification of linear peptides present in the three dimensional structure of the virus involved. In addition the human polyoma virus peptide sequences can be used for B-cell stimulation and/or B-cell functionality studies.

The human polyoma virus peptide sequences of the invention can also be part of a device or kit further containing means for measuring antibodies in a human test sample, like serum, plasma or whole blood.

In addition, the human polyoma virus peptide sequences mentioned in Table 9 can be used, directly or indirectly, for the manufacture of a medicament to treat progressive multifocal leukoencephalopathy (PML).

### EXPERIMENTAL SECTION

A peptide array representing human polyoma virus proteins has been prepared. The following proteins are covered by the peptide array: agnoprotein, small T antigen, large T antigen, VP1, VP2, VP3 and VP4 of the viruses BK, JC, KI, WU, MC and SV40. In addition, the VP1 protein of the viruses HPyV6, HPyV7, HPyV9, IPPyV and TSV are also included in this study. In total 4284 15-mer peptides overlapping by 11 residues are displayed in triplicates on one single array chip.

In order to prepare the peptide microarrays, polyoma virus protein sequences were retrieved from the NCBI (National Center for Biotechnology) database. The best covering sequence for each of the proteins of each virus was calculated. Then, each sequence was divided in all possible 15-mer peptides and coverage of related sequences by the peptides was calculated. The protein sequence providing the best covering peptides was determined.

Mosaic sequences, which further increase the coverage of related sequences, were generated as well. The mosaic algorithm assembles artificial best covering sequences for a given sequence pool. The number of sequences that were retrieved from the NCBI database is given in Table 1 and Table 2.

For the design of the 15-mer peptides, the following proteins were included:
Agnoprotein: 3 best covering sequences, one from each of the viruses BK, JC, SV40 and 6 mosaic sequences
large T antigen: 6 best covering sequences, one from each of the viruses: BK, JC, KI, MC, SV40, WU and 2 mosaic sequences
small T antigen: 6 best covering sequences, one from each of the viruses: BK, JC, KI, MC, SV40, WU and 2 mosaic sequences
VP1: All available sequences from the viruses: BK, JC, KI, MC, SV40, WU, HPyV6, HPyV7, HPyV9, IPPyV and TSV
VP2: 6 best covering sequences, one from each of the viruses: BK, JC, KI, MC, SV40, WU and 2 mosaic sequences
VP3: 6 best covering sequences, one from each of the viruses: BK, JC, KI, MC, SV40, WU and 2 mosaic sequences
VP4: The one available sequence from SV40

### Clinical Samples Used:

A total of 49 plasma samples from healthy volunteers (HV) have been tested on the peptide microarrays.

### Analysis:

Peptides from the microarray that were reactive against antibodies present in the HV plasma samples were aligned against consensus sequences retrieved from the NCBI database. Table 3 provides the accession numbers for the sequences used in the analysis. For analysis purposes, the different proteins for the different organisms were labeled with a unique code (ID). Table 4 gives an overview of these unique identifiers.

### Results

### Overview of the Hybridization Results.

A total of 49 clinical samples were tested on the peptide microarrays in triplicate (each peptide array contains 3 identical subarrays of 4284 peptides). Data from the subarrays were pooled, and only the median value (in case of 3 valid subarray data points), or the average of 2 data points (in case one of the subarray data points was excluded for quality reasons) were retained for further analysis. This will result in 209,916 data points (4284×49).

As a negative control, hybridization buffer without addition of human plasma was run alongside. Analysis of these 4284 control data points showed the following boxplot parameters:
Minimum=507 fluorescent units (FU; relative measure, equipment dependent)
25th quartile=590 FU
Median=614 FU
75th quartile=642 FU
Maximum=15859 FU

For further analysis, the value of the 75th quartile is used as a cut-off, because it is reasonable to assume that from that moment onwards meaningful biological data might be available with the HV samples.

The following arbitrary classes of signal intensity were generated and represented in Table 5:
a. FU signal >642, but <=10,000
b. FU signal >10,000, but <=20,000
c. FU signal >20,000, but <=30,000
d. FU signal >30,000

The most important results are found in the FU group of >30,000, with a total of 1,148 data points. However, the presentation of this result does not educate on the number of peptides that are responsible for this hybridization signal. Therefore, a further analysis of these data points was needed (given in Table 6).

A total of 635 peptides are responsible for the 1148 data points with an FU value >30,000. The 635 peptides are distributed over different classes of organisms and genes, with strong response to small T antigen peptides being the most prevalent for KIV, WUV, MCV, and JCV, followed by large T antigen and VP1, and a strong signal is the least prevalently found in VP2, VP3, and Agnoprotein. The sequence of these 635 peptides is given in Table 19. For interpretation of the origin of the peptides see Table 20

IDs given in table 19 which are not defined in table 20 do not represent further specified polyoma virus peptide sequences.

### Immunodominancy

Subsequently, an analysis towards the immuno-dominancy of these peptides was conducted. Therefore, for each of the 4284 peptides the number of hits was searched for with a FU of >10,000 in each of the 49 HV samples.

The analysis retrieved the following result: 2424 peptides had at least “one out of the 49” HV samples a FU-value >10,000. As a consequence, 1860 peptides were having FU values below the arbitrary cut-off of 10,000 for all the samples tested (Note: this does not mean that for certain disease states these peptides might not show reaction with available antibodies). In addition, subgroups of prevalence were defined in blocks of 5 HV (Table 7). For the purpose of this exercise, we considered reaction on a peptide as immunodominant from >21 reactions (out of 49 HV) onwards.

A total of 63 peptides were identified for which the label of immunodominant epitope would be applicable (according to the above assumptions) (Table 8). The sequence of these 63 immuno dominant peptides is given in Table 9.

Detection of Peptides with Average FU Values >10000 Across the 49 HV

The dataset of 209,916 data points was analyzed for average values per peptide. This means that for each peptide, the average of FU values was calculated across the 49 HV reaction patterns. A total of 106 peptides were retrieved with values >10,000. The distribution of these peptides per organism is given in Table 10. In Table 11 to 18 the peptide sequences per organism are given.

### SUMMARY

Peptide arrays (15-mer peptides) were prepared covering all proteins of human polyoma viruses including BK virus, JC virus, KI virus, WU virus, MC virus, SV40, HPyV6, HPyV7, HPyV9, IPPyV and TSV.

Serum samples from 49 healthy volunteers were tested for the presence of antibodies against these peptides. As a result a set of potential B-cell epitopes were identified as described above.

TABLE 1
Number of protein sequences retrieved from the NCBI database for
the indicated viruses

| | agno | large T | small T | VP1 | VP2 | VP3 | VP4 |
|---|---|---|---|---|---|---|---|
| BKV | 305 | 381 | 339 | 1338 | 295 | 289 | |
| JCV | 710 | 1993 | 638 | 2481 | 642 | 638 | |
| KIV | | 13 | 30 | 53 | 12 | 9 | |
| MCV | | 110 | 65 | 60 | 34 | 11 | |
| SV40 | 51 | 149 | 53 | 60 | 51 | 29 | 1 |
| WUV | | 90 | 85 | 84 | 223 | 73 | |

TABLE 2
Number of protein sequences retrieved from NCBI database for other
polyoma viruses

| | agno | large T | small T | VP1 | VP2 | VP3 | VP4 |
|---|---|---|---|---|---|---|---|
| HPyV6 | | 7 | 7 | 7 | 7 | 7 | |
| HPyV7 | | 7 | 7 | 7 | 7 | 7 | |
| HPyV9 | | 2 | 2 | 2 | 2 | 2 | |
| IPPyV | | 1 | 1 | 1 | 1 | 1 | |
| TSV | | 2 | 2 | 2 | 2 | 2 | |

TABLE 3
NCBI database accession numbers for polyomavirus proteins used in the peptide analysis.

| | ACCESSION | | | | | | | |
|---|---|---|---|---|---|---|---|---|
| | NUMBER | | | | | | | |
| | BKV | JCV | KIV | MCV | SV40 | WUV | HPyV6 | HPyV7 |
| VP1 | CAA24299 | AAA82101 | ACB12026 | AEM01098 | YP_003708381 | ACB12036 | YP_003848918 | YP_003848923 |
| VP2 | | AAA82099 | ACB12024 | AEM01099 | YP_003708379 | ACB12034 | | |
| large | CAA24300 | AAA82102 | ACB12028 | AEM01097 | YP_003708382 | ACB12038 | | |
| T | | | | | | | | |
| small | CAA24301 | AAA82103 | ACB12027 | AEM01096 | YP_003708383 | ACB12037 | | |
| T | | | | | | | | |

TABLE 4
Protein and organism identifier (ID)

| | ID | ID | ID | ID | ID | ID | ID | |
|---|---|---|---|---|---|---|---|---|
| | BKV | JCV | KIV | MCV | SV40 | WUV | HPyV6 | ID HPyV7 |
| VP1 | _1_01 | _1_02 | _1_03 | _1_04 | _1_05 | _1_06 | _1_09 | _1_10 |
| VP2 | _2_01 | _2_02 | _2_03 | _2_04 | _2_05 | _2_06 | _2_09 | _2_10 |
| large T | _4_01 | _4_02 | _4_03 | _4_04 | _4_05 | _4_06 | _4_09 | _4_10 |
| small T | _5_01 | _5_02 | _5_03 | _5_04 | _5_05 | _5_06 | _5_09 | _5_10 |

TABLE 5
Overview of the different FU classes per organism and per viral protein.

| | | | | Fluorescent units | | | | |
|---|---|---|---|---|---|---|---|---|
| | | | n | >642 | >10000 | >20000 | | |
| Organism | gene | ID | peptides | <10000 | <20000 | <30000 | >30000 | total |
| | other | 0_05 | 3 | 146 | — | — | 1 | 147 |
| other | VP1 | 1_00 | 467 | 22,056 | 622 | 112 | 93 | 22,883 |
| BKV | VP1 | 1_01 | 423 | 20,146 | 461 | 60 | 60 | 20,727 |
| JCV | VP1 | 1_02 | 758 | 35,160 | 1,518 | 264 | 200 | 37,142 |
| KIV | VP1 | 1_03 | 69 | 3,295 | 70 | 8 | 8 | 3,381 |
| MCV | VP1 | 1_04 | 165 | 7,774 | 246 | 34 | 31 | 8,085 |
| SV40 | VP1 | 1_05 | 69 | 3,300 | 59 | 9 | 13 | 3,381 |
| WUV | VP1 | 1_06 | 83 | 3,980 | 69 | 11 | 7 | 4,067 |
| IPPyV | VP1 | 1_07 | 89 | 4,147 | 166 | 26 | 22 | 4,361 |
| TSV | VP1 | 1_08 | 89 | 4,105 | 180 | 39 | 37 | 4,361 |
| HPyV6 | VP1 | 1_09 | 97 | 4,482 | 180 | 40 | 51 | 4,753 |
| HPyV7 | VP1 | 1_10 | 115 | 5,391 | 191 | 31 | 22 | 5,635 |
| BKV | VP2 | 2_01 | 81 | 3,860 | 91 | 10 | 8 | 3,969 |
| JCV | VP2 | 2_02 | 71 | 3,276 | 155 | 19 | 29 | 3,479 |
| KIV | VP2 | 2_03 | 96 | 4,564 | 99 | 22 | 19 | 4,704 |
| MCV | VP2 | 2_04 | 57 | 2,703 | 66 | 9 | 15 | 2,793 |
| SV40 | VP2 | 2_05 | 74 | 3,473 | 119 | 20 | 14 | 3,626 |
| WUV | VP2 | 2_06 | 92 | 4,356 | 105 | 23 | 24 | 4,508 |
| mosaic | VP2 | 2_12 | 68 | 3,200 | 65 | 19 | 48 | 3,332 |
| JCV | VP3 | 3_02 | 3 | 147 | — | — | — | 147 |
| MCV | VP3 | 3_04 | 6 | 292 | 1 | — | 1 | 294 |
| BKV | large T | 4_01 | 162 | 7,624 | 223 | 54 | 37 | 7,938 |
| JCV | large T | 4_02 | 136 | 6,323 | 277 | 36 | 28 | 6,664 |
| KIV | large T | 4_03 | 157 | 7,194 | 374 | 70 | 55 | 7,693 |
| MCV | large T | 4_04 | 202 | 9,423 | 345 | 64 | 66 | 9,898 |
| SV40 | large T | 4_05 | 155 | 7,119 | 357 | 60 | 59 | 7,595 |
| WUV | large T | 4_06 | 155 | 7,040 | 384 | 96 | 75 | 7,595 |
| mosaic | large T | 4_12 | 75 | 3,487 | 149 | 20 | 19 | 3,675 |
| BKV | small T | 5_01 | 21 | 925 | 83 | 13 | 8 | 1,029 |
| JCV | small T | 5_02 | 22 | 935 | 103 | 25 | 15 | 1,078 |
| KIV | small T | 5_03 | 27 | 1,068 | 202 | 35 | 18 | 1,323 |
| MCV | small T | 5_04 | 27 | 1,163 | 124 | 24 | 12 | 1,323 |
| SV40 | small T | 5_05 | 24 | 1,067 | 90 | 14 | 5 | 1,176 |
| WUV | small T | 5_06 | 28 | 1,153 | 167 | 35 | 17 | 1,372 |
| mosaic | small T | 5_12 | 24 | 939 | 170 | 42 | 25 | 1,176 |
| BKV | agno | 6_01 | 13 | 624 | 10 | 1 | 2 | 637 |
| JCV | agno | 6_02 | 15 | 725 | 9 | 1 | — | 735 |
| SV40 | agno | 6_05 | 13 | 611 | 25 | — | 1 | 637 |
| mosaic | agno | 6_12 | 53 | 2,561 | 26 | 7 | 3 | 2,597 |
| | | TOTAL | 4284 | 199,834 | 7,581 | 1,353 | 1,148 | 209,916 |

TABLE 6
Identification of organism_gene peptides with FU value >30,000.

| | | | | | | % |
|---|---|---|---|---|---|---|
| | | | | number of | n peptides | peptides |
| | | | | hits with | with | with |
| | | | n | FU value | FU value | FU value |
| organism | gene | ID | peptides | >30000 | >30000 | >30000 |
| JCV | VP3 | 3_02 | 3 | 0 | 0 | 0 |
| JCV | agno | 6_02 | 15 | 0 | 0 | 0 |
| BKV | VP2 | 2_01 | 81 | 8 | 4 | 5 |
| mosaic | agno | 6_12 | 53 | 3 | 3 | 6 |
| WUV | VP1 | 1_06 | 83 | 7 | 5 | 6 |
| SV40 | agno | 6_05 | 13 | 1 | 1 | 8 |
| SV40 | VP1 | 1_05 | 69 | 13 | 6 | 9 |
| BKV | VP1 | 1_01 | 423 | 60 | 40 | 9 |
| HPyV7 | VP1 | 1_10 | 115 | 22 | 11 | 10 |
| KIV | VP1 | 1_03 | 69 | 8 | 7 | 10 |
| MCV | VP2 | 2_04 | 57 | 15 | 6 | 11 |
| mosaic | VP2 | 2_12 | 68 | 48 | 8 | 12 |
| SV40 | VP2 | 2_05 | 74 | 14 | 9 | 12 |
| other | VP1 | 1_00 | 467 | 93 | 57 | 12 |
| KIV | VP2 | 2_03 | 96 | 19 | 13 | 14 |
| JCV | VP2 | 2_02 | 71 | 29 | 10 | 14 |
| MCV | large T | 4_04 | 202 | 66 | 29 | 14 |
| MCV | VP1 | 1_04 | 165 | 31 | 25 | 15 |
| JCV | VP1 | 1_02 | 758 | 200 | 116 | 15 |
| BKV | agno | 6_01 | 13 | 2 | 2 | 15 |
| BKV | large T | 4_01 | 162 | 37 | 25 | 15 |
| JCV | large T | 4_02 | 136 | 28 | 21 | 15 |
| WUV | VP2 | 2_06 | 92 | 24 | 15 | 16 |
| MCV | VP3 | 3_04 | 6 | 1 | 1 | 17 |
| SV40 | small T | 5_05 | 24 | 5 | 4 | 17 |
| IPPyV | VP1 | 1_07 | 89 | 22 | 15 | 17 |
| HPyV6 | VP1 | 1_09 | 97 | 51 | 18 | 19 |
| mosaic | large T | 4_12 | 75 | 19 | 14 | 19 |
| BKV | small T | 5_01 | 21 | 8 | 4 | 19 |
| KIV | large T | 4_03 | 157 | 55 | 30 | 19 |
| SV40 | large T | 4_05 | 155 | 59 | 31 | 20 |
| WUV | large T | 4_06 | 155 | 75 | 35 | 23 |
| JCV | small T | 5_02 | 22 | 15 | 5 | 23 |
| TSV | VP1 | 1_08 | 89 | 37 | 26 | 29 |
| MCV | small T | 5_04 | 27 | 12 | 8 | 30 |
| WUV | small T | 5_06 | 28 | 17 | 9 | 32 |
| | other | 0_05 | 3 | 1 | 1 | 33 |
| KIV | small T | 5_03 | 27 | 18 | 10 | 37 |
| mosaic | small T | 5_12 | 24 | 25 | 11 | 46 |
| | | | | 1148 | 635 | |

TABLE 7
Detection of immunodominant epitopes.

| | | | Total | | | | | | | | | | | | total |
|---|---|---|---|---|---|---|---|---|---|---|---|---|---|---|---|
| | | | Peptides | number of HV samples that show reactivity | | | | | | | | | | | >1 |
| | | | in | | | >6 | >11 | >16 | >21 | >26 | >31 | >36 | >41 | >46 | sample |
| Organism | gene | ID | class | 0 | <=5 | <=10 | <=15 | <=20 | <=25 | <=30 | <=35 | <=40 | <=45 | <=49 | reactive |
| | other | 0_05 | 3 | 2 | 1 | 0 | 0 | 0 | 0 | 0 | 0 | 0 | 0 | 0 | 1 |
| other | VP1 | 1_00 | 467 | 245 | 168 | 38 | 8 | 4 | 2 | 0 | 1 | 0 | 1 | 0 | 222 |
| BKV | VP1 | 1_01 | 423 | 206 | 190 | 20 | 5 | 0 | 1 | 0 | 0 | 1 | 0 | 0 | 217 |
| JCV | VP1 | 1_02 | 758 | 327 | 301 | 81 | 22 | 15 | 10 | 1 | 0 | 1 | 0 | 0 | 431 |
| KIV | VP1 | 1_03 | 69 | 33 | 33 | 2 | 1 | 0 | 0 | 0 | 0 | 0 | 0 | 0 | 36 |
| MCV | VP1 | 1_04 | 165 | 78 | 65 | 16 | 5 | 0 | 1 | 0 | 0 | 0 | 0 | 0 | 87 |
| SV40 | VP1 | 1_05 | 69 | 41 | 23 | 4 | 1 | 0 | 0 | 0 | 0 | 0 | 0 | 0 | 28 |
| WUV | VP1 | 1_06 | 83 | 43 | 37 | 1 | 2 | 0 | 0 | 0 | 0 | 0 | 0 | 0 | 40 |
| IPPyV | VP1 | 1_07 | 89 | 20 | 53 | 13 | 2 | 1 | 0 | 0 | 0 | 0 | 0 | 0 | 69 |
| TSV | VP1 | 1_08 | 89 | 18 | 53 | 14 | 3 | 1 | 0 | 0 | 0 | 0 | 0 | 0 | 71 |
| HPyV6 | VP1 | 1_09 | 97 | 35 | 46 | 10 | 2 | 2 | 2 | 0 | 0 | 0 | 0 | 0 | 62 |
| HPyV7 | VP1 | 1_10 | 115 | 48 | 51 | 12 | 1 | 2 | 0 | 1 | 0 | 0 | 0 | 0 | 67 |
| BKV | VP2 | 2_01 | 81 | 41 | 34 | 4 | 2 | 0 | 0 | 0 | 0 | 0 | 0 | 0 | 40 |
| JCV | VP2 | 2_02 | 71 | 32 | 25 | 7 | 3 | 3 | 1 | 0 | 0 | 0 | 0 | 0 | 39 |
| KIV | VP2 | 2_03 | 96 | 58 | 29 | 6 | 1 | 1 | 0 | 1 | 0 | 0 | 0 | 0 | 38 |
| MCV | VP2 | 2_04 | 57 | 36 | 13 | 6 | 1 | 0 | 1 | 0 | 0 | 0 | 0 | 0 | 21 |
| SV40 | VP2 | 2_05 | 74 | 41 | 22 | 8 | 1 | 1 | 1 | 0 | 0 | 0 | 0 | 0 | 33 |
| WUV | VP2 | 2_06 | 92 | 43 | 42 | 6 | 0 | 0 | 0 | 0 | 1 | 0 | 0 | 0 | 49 |
| mosaic | VP2 | 2_12 | 68 | 33 | 28 | 3 | 1 | 3 | 0 | 0 | 0 | 0 | 0 | 0 | 35 |
| JCV | VP3 | 3_02 | 3 | 3 | | | | | | | | | | | |
| MCV | VP3 | 3_04 | 6 | 4 | 2 | 0 | 0 | 0 | 0 | 0 | 0 | 0 | 0 | 0 | 2 |
| BKV | large T | 4_01 | 162 | 67 | 70 | 20 | 3 | 0 | 1 | 0 | 1 | 0 | 0 | 0 | 95 |
| JCV | large T | 4_02 | 136 | 50 | 64 | 14 | 3 | 4 | 1 | 0 | 0 | 0 | 0 | 0 | 86 |
| KIV | large T | 4_03 | 157 | 49 | 78 | 19 | 5 | 2 | 1 | 2 | 0 | 0 | 1 | 0 | 108 |
| MCV | large T | 4_04 | 202 | 74 | 99 | 19 | 6 | 2 | 1 | 0 | 0 | 0 | 0 | 1 | 128 |
| SV40 | large T | 4_05 | 155 | 63 | 61 | 18 | 3 | 5 | 3 | 2 | 0 | 0 | 0 | 0 | 92 |
| WUV | large T | 4_06 | 155 | 54 | 71 | 18 | 2 | 3 | 2 | 2 | 1 | 1 | 1 | 0 | 101 |
| mosaic | large T | 4_12 | 75 | 27 | 39 | 4 | 2 | 3 | 0 | 0 | 0 | 0 | 0 | 0 | 48 |
| BKV | small T | 5_01 | 21 | 6 | 5 | 6 | 3 | 1 | 0 | 0 | 0 | 0 | 0 | 0 | 15 |
| JCV | small T | 5_02 | 22 | 7 | 6 | 4 | 2 | 2 | 0 | 0 | 0 | 1 | 0 | 0 | 15 |
| KIV | small T | 5_03 | 27 | 4 | 8 | 6 | 3 | 2 | 0 | 2 | 1 | 1 | 0 | 0 | 23 |
| MCV | small T | 5_04 | 27 | 8 | 8 | 6 | 2 | 0 | 1 | 2 | 0 | 0 | 0 | 0 | 19 |
| SV40 | small T | 5_05 | 24 | 3 | 12 | 5 | 3 | 1 | 0 | 0 | 0 | 0 | 0 | 0 | 21 |
| WUV | small T | 5_06 | 28 | 2 | 15 | 3 | 3 | 1 | 2 | 1 | 0 | 0 | 0 | 1 | 26 |
| mosaic | small T | 5_12 | 24 | 2 | 5 | 6 | 4 | 4 | 2 | 1 | 0 | 0 | 0 | 0 | 22 |
| BKV | agno | 6_01 | 13 | 9 | 3 | 1 | 0 | 0 | 0 | 0 | 0 | 0 | 0 | 0 | 4 |
| JCV | agno | 6_02 | 15 | 9 | 6 | 0 | 0 | 0 | 0 | 0 | 0 | 0 | 0 | 0 | 6 |
| SV40 | agno | 6_05 | 13 | 5 | 6 | 2 | 0 | 0 | 0 | 0 | 0 | 0 | 0 | 0 | 8 |
| mosaic | agno | 6_12 | 53 | 34 | 18 | 1 | 0 | 0 | 0 | 0 | 0 | 0 | 0 | 0 | 19 |
| | | Total | | | 1790 | 403 | 105 | 63 | 33 | 15 | 5 | 5 | 3 | 2 | 2424 |

TABLE 8
Distribution of 63 immuno dominant peptides

| | BKV | JCV | KIV | MCV | SV40 | WUV | HPyV6 | HPyV7 | mosaic | else | total |
|---|---|---|---|---|---|---|---|---|---|---|---|
| VP1 | 2 | 12 | | 1 | | | 2 | 1 | | 4 | 22 |
| VP2 | | 1 | 1 | 1 | 1 | 1 | | | | | 5 |
| large T | 2 | 1 | 4 | 2 | 5 | 7 | | | | | 21 |
| small T | | 1 | 4 | 3 | | 7 | | | | | 15 |
| total | 4 | 15 | 9 | 7 | 6 | 15 | 2 | 1 | 0 | 4 | 63 |

TABLE 9
Sequences of the 63 immuno dominant peptides

| | | | peptide | |
|---|---|---|---|---|
| ID | organism | gene | number | peptide sequence |
| 4_01 | BKV | large T | 3118 | LDSEISMYTFSRMKY |
| 4_01 | BKV | large T | 3119 | ISMYTFSRMKYNICM |
| 4_02 | JCV | large T | 3244 | TMNEYSVPRTLQARF |
| 4_03 | KIV | large T | 3332 | KGVNNPYGLYSRMCR |
| 4_03 | KIV | large T | 3295 | IPTYGTPDWDEWWSQ |
| 4_03 | KIV | large T | 3279 | MSCWGNLPLMRRQYL |
| 4_03 | KIV | large T | 3333 | NPYGLYSRMCRQPFN |
| 4_04 | MCV | large T | 3546 | LAHYLDFAKPFPCQK |
| 4_04 | MCV | large T | 3532 | MPEMYNNLCKPPYKL |
| 4_05 | SV40 | large T | 3637 | LGLERSAWGNIPLMR |
| 4_05 | SV40 | large T | 3723 | MVYNIPKKRYWLFKG |
| 4_05 | SV40 | large T | 3638 | RSAWGNIPLMRKAYL |
| 4_05 | SV40 | large T | 3784 | HNQPYHICRGFTCFK |
| 4_05 | SV40 | large T | 3653 | PTYGTDEWEQWWNAF |
| 4_06 | WUV | large T | 3810 | GTPDWDYWWSQFNSY |
| 4_06 | WUV | large T | 3932 | LIWCRPVSDFHPCIQ |
| 4_06 | WUV | large T | 3792 | LGLDMTCWGNLPLMR |
| 4_06 | WUV | large T | 3919 | TMNEYLVPATLAPRF |
| 4_06 | WUV | large T | 3920 | YLVPATLAPRFHKTV |
| 4_06 | WUV | large T | 3809 | IPTYGTPDWDYWWSQ |
| 4_06 | WUV | large T | 3793 | MTCWGNLPLMRTKYL |
| 5_02 | JCV | small T | 4054 | IDCYCFDCFRQWFGC |
| 5_03 | KIV | small T | 4079 | KPPVWIECYCYKCYR |
| 5_03 | KIV | small T | 4063 | QSSQVYCKDLCCNKF |
| 5_03 | KIV | small T | 4075 | HCILSKYHKEKYKIY |
| 5_03 | KIV | small T | 4072 | CIHGYNHECQCIHCI |
| 5_04 | MCV | small T | 4105 | KQKNCLTWGECFCYQ |
| 5_04 | MCV | small T | 4094 | DYMQSGYNARFCRGP |
| 5_04 | MCV | small T | 4095 | SGYNARFCRGPGCML |
| 5_06 | WUV | small T | 4159 | WIECYCYRCYREWFG |
| 5_06 | WUV | small T | 4154 | YCFLDKRHKQKYKIF |
| 5_06 | WUV | small T | 4143 | ELCCNFPPRKYRLVG |
| 5_06 | WUV | small T | 4158 | KPPMWIECYCYRCYR |
| 5_12 | WUV | small T | 4172 | FGTWNSSEVSCDFPP |
| 5_12 | WUV | small T | 4187 | PLCPDTLYCKDWPIC |
| 5_12 | WUV | small T | 4190 | IDCYCFDCFRQWFGL |
| 1_01 | BKV | VP1 | 531 | EKKMLPCYSTARIPL |
| 1_01 | BKV | VP1 | 791 | LPCYSTARIPLPNLY |
| 1_09 | HPyV6 | VP1 | 2306 | AAGAANLFGPPVEKQ |
| 1_09 | HPyV6 | VP1 | 2289 | TVDMMFRQFLQPQKP |
| 1_10 | HPyV7 | VP1 | 2404 | ATTGNFQSRGLPYPM |
| 1_02 | JCV | VP1 | 929 | DPDMMRYVDRYGQLQ |
| 1_02 | JCV | VP1 | 1576 | FNYRTMYPDGTIFPK |
| 1_02 | JCV | VP1 | 1562 | FNYRTTYPHGTIFPK |
| 1_02 | JCV | VP1 | 956 | GMFTNRCGSQQWRGL |
| 1_02 | JCV | VP1 | 1177 | GMFTNRSGFQQWRGL |
| 1_02 | JCV | VP1 | 958 | MRYVDRYGQLQTQML |
| 1_02 | JCV | VP1 | 974 | PDMMRYVDRYGQSQT |
| 1_02 | JCV | VP1 | 927 | PGDPDMMRYVDRYGQ |
| 1_02 | JCV | VP1 | 1338 | PNLNEDLTCGNIPMW |
| 1_02 | JCV | VP1 | 1528 | YLYKNKAYPVECWVP |
| 1_02 | JCV | VP1 | 926 | LPGDPDMMRYVDRYG |
| 1_02 | JCV | VP1 | 1427 | GMFTNRSCSQQWRGL |
| 1_04 | MCV | VP1 | 1817 | AKLDKDGNYPIEVWC |
| 1_00 | other | VP1 | 99 | PDMMRYVDKYGQLQT |
| 1_00 | other | VP1 | 352 | WVADPSRNDNCRYFG |
| 1_00 | other | VP1 | 237 | KAYLDKNNAYPVECW |
| 1_00 | other | VP1 | 285 | PLEMQGVLMNYRTKY |
| 2_02 | JCV | VP2 | 2538 | AFVNNIHYLDPRHWG |
| 2_03 | KIV | VP2 | 2616 | YQLETGIPGIPDWLF |
| 2_04 | MCV | VP2 | 2707 | MAFSLDPLQWENSLL |
| 2_05 | SV40 | VP2 | 2754 | MAVDLYRPDDYYDIL |
| 2_06 | WUV | VP2 | 2837 | YNLETGIPGVPDWVF |

TABLE 10
Distribution of 106 peptides with average signal >10000

| | BKV | JCV | KIV | MCV | SV40 | WUV | HPyV6 | HPyV7 | mosaic | else | total |
|---|---|---|---|---|---|---|---|---|---|---|---|
| VP1 | 2 | 20 | 0 | 1 | 1 | 0 | 6 | 2 | | 6 | 38 |
| VP2 | 0 | 3 | 2 | 1 | 2 | 1 | 0 | 0 | 4 | | 13 |
| large T | 2 | 2 | 5 | 3 | 9 | 9 | 0 | 0 | 1 | | 31 |
| small T | 0 | 3 | 5 | 3 | 1 | 7 | 0 | 0 | 5 | | 24 |
| total | 4 | 28 | 12 | 8 | 13 | 17 | 6 | 2 | 10 | 6 | 106 |

TABLE 11
Peptides for JCV

| ACCESSION | | peptide | aa | peptide | alignment |
|---|---|---|---|---|---|
| NUMBER | | number | position | sequence | |
| AAA82102 | Large | 3244 | 528-542 | TMNEYSVPRTLQARF | |
| | T-JCV | | | | |
| | Large | 3271 | 666-680 | EHCTYHICKGFQCFK | |
| | T-JCV | | | | |
| | | | | | FGTWNSSEVGCDFPP |
| AAA82103 | Small | 4040 | 74-88 | FGTWNSSEVGCDFPP | -------------- |
| | T-JCV | | | | |
| | Small | 4185 | 74-88 | FGTWNSSEVCADFPL | ---------CA---L |
| | mosaic | | | | |
| | T-Small | 4172 | 74-88 | FGTWNSSEVSCDFPP | ---------S----- |
| | T-mosaic | | | | |
| | | | | | HCPCLMCMLKLRHKNRKFL |
| | Small | 4174 | 108-122 | HCPCLMCMLKLRHKN | --------------- |
| | T-mosaic | | | | |
| | Small | 4049 | 112-126 | LMCMLKLRHRNRKFL | ---------R----- |
| | T-JCV | | | | |
| | Small | 4175 | 112-126 | LMCMLKLRHKNRKFL | --------------- |
| | T-mosaic | | | | |
| | | | | | IDCYCFDCFRQWFGC |
| | Small | 4054 | 134-148 | IDCYCFDCFRQWFGC | --------------- |
| | T-JCV | | | | |
| | Small | 4190 | 134-148 | IDCYCFDCFRQWFGL | --------------L |
| | T-mosaic | | | | |
| AAA82101 | VP1-else | 209 | 72-86 | SPERKMLPCYSTARI | |
| | VP1-JCV | 1338 | 89-103 | PNLNEDLTCGNIPMW | |
| | | | | | ALELQGVVCNYRTKYPDGTIFPK |
| | VP1-JCV | 1445 | 151-165 | ALELQGVVCNYRTKY | --------------- |
| | VP1-else | 285 | 151-165 | PLEMQGVLMNYRTKY | P--M---LM------ |
| | (JCV) | | | | |
| | VP1-JCV | 1576 | 159-173 | FNYRTMYPDGTIFPK | F----M--------- |
| | VP1-JCV | 1562 | 159-173 | FNYRTTYPHGTIFPK | F----T--H------ |
| | | | | | KAYLDKNNAYPVECWVP |
| | VP1-else | 237 | 187-201 | KAYLDKNNAYPVECW | --------------- |
| | VP1-JCV | 1568 | 189-203 | YLDENKAYPVECWVP | ---E-K--------- |
| | VP1-JCV | 1646 | 189-203 | YLDRNKAYPVECWVP | ---R-K--------- |
| | VP1-JCV | 1528 | 189-203 | YLYKNKAYPVECWVP | --Y--K--------- |
| | VP1-JCV | 1133 | 234-248 | TTVLLDEFGGGPLCK | |
| | VP1-JCV | 1146 | 234-248 | TTVLLDEYGVGPLCK | |
| | VP1-JCV | 1015 | 241-255 | FGVGPLCKGANLYLS | |
| | | | | | GMFTNRCGSQQWRGL |
| | VP1-JCV | 956 | 261-275 | GMFTNRCGSQQWRGL | --------------- |
| | VP1-JCV | 1427 | 261-275 | GMFTNRSCSQQWRGL | ------SC------- |
| | VP1-JCV | 1177 | 261-275 | GMFTNRSGFQQWRGL | ------SGF------ |
| | | | | | LPGDPDMMRYVDRYGQLQTQML |
| | VP1-JCV | 926 | 333-347 | LPGDPDMMRYVDRYG | --------------- |
| | VP1-JCV | 927 | 334-348 | PGDPDMMRYVDRYGQ | --------------- |
| | VP1-JCV | 1649 | 335-349 | GDPDMMRYVDSCRQK | ----------SCR-K |
| | VP1-JCV | 929 | 336-350 | DPDMMRYVDRYGQLQ | --------------- |
| | VP1-else | 99 | 337-351 | PDMMRYVDKYGQLQT | --------K------ |
| | VP1-JCV | 974 | 337-351 | PDMMRYVDRYGQSQT | ------------S-- |
| | VP1-JCV | 957 | 338-352 | DMMRYVDRYGQLQTQ | --------------- |
| | VP1-JCV | 958 | 340-354 | MRYVDRYGQLQTQML | --------------- |
| AAA82099 | VP2-mosaic | 2909 | 116-130 | QQPVMALQLFNPEDY | |
| | VP2-JCV | 2538 | 140-154 | AFVNNIHYLDPRHWG | |
| | | | | | NLVRDDLPSLTSQEIQRRT |
| | VP2-JCV | 2544 | 167-181 | NLVRDDLPSLTSQEI | -------------- |
| | VP2-mosaic | 2911 | 167-181 | NLVRDDLPALTSQEI | --------A------ |
| | VP2-mosaic | 2940 | 167-181 | NLVRDDLPSLTSREI | ------------R-- |
| | VP2-mosaic | 2941 | 171-185 | DDLPSLTSREIQRRT | --------R------ |
| | VP2-JCV | 2572 | 286-300 | ANQRSAPQWMLPLLL | |

TABLE 12
Peptides for BKV

| ACC | | aa | peptide | peptide sequence |
|---|---|---|---|---|
| NUMBERS | gene | position | sequence | aligned |
| CAA24300 | Large T-BKV | 605-619 | LDSEISMYTFSRMKY | LDSEISMYTFSRMKY |
| | Large T-BKV | 609-623 | ISMYTFSRMKYNICM | -----------NICM |
| | Large T-mosaic | 231-245 | EYLLYSALTRDPYYI | |
| | (Bkvirus) | | | |
| CAA24301 | Small T-mosaic | 74-88 | FGTWNSSEVCADFPL | FGTWNSSEVCADFPL |
| | Small T-mosaic | 74-88 | FGTWNSSEVSCDFPP | ---------SC---P |
| | Small T-mosaic | 108-122 | HCPCLMCMLKLRHKN | |
| | Small T-mosaic | 134-148 | IDCYCFDCFRQWFGL | |
| | | | | SPERKMLPCYSTARIPLPNLY |
| CAA24299 | VP1-else (BKV) | 80-94 | SPERKMLPCYSTARI | --------------- |
| | VP1-BKV | 82-96 | EKKMLPCYSTARIPL | -K------------- |
| | VP1-BKV | 86-100 | LPCYSTARIPLPNLY | --------------- |
| | VP1-else (BKV) | 195-209 | KAYLDKNNAYPVECW | |

TABLE 13
Peptides for KIV

| | | aa | |
|---|---|---|---|
| ACC NUMBERS | | position | ID |
| ACB12028 | Large T-KIV | 21-35 | MSCWGNLPLMRRQYL |
| | Large T-KIV | 85-99 | IPTYGTPDWDEWWSQ |
| | Large T-KIV | 233-247 | KGVNNPYGLYSRMCR |
| | Large T-KIV | 237-251 | NPYGLYSRMCRQPFN |
| | Large T-KIV | 269-283 | EDLFGEPKEPSLSWN |
| ACB12027 | Small T-KIV | | CIHGYNHECQCIHCI |
| | Small T-KIV | | HCILSKYHKEKYKIY |
| | Small T-KIV | | KPPVWIECYCYKCYR |
| | Small T-KIV | | QSSQVYCKDLCCNKF |
| | Small T-KIV | | VYCKDLCCNKFRLVG |
| ACB12026 | VP1-else | 112-126 | PDIPNQVSECDMLIW |
| | (WUV, KIV) | | |
| | VP1-else | 219-233 | WVADPSRNDNCRYFG |
| | (WUV, KIV) | | |
| ACB12024 | VP2-KIV | 317-331 | TGGTPHYATPDWILY |
| | VP2-KIV | 152-166 | YQLETGIPGIPDWLF |

TABLE 14
Peptides for MCV

| acc number | | as position | ID |
|---|---|---|---|
| AEM01097 | Large T-MCV | 405-419 | MPEMYNNLCKPPYKL |
| AEM01097 | Large T-MCV | 413-427 | CKPPYKLLQENKPLL |
| AEM01097 | Large T-MCV | 461-475 | LAHYLDFAKPFPCQK |
| AEM01096 | Small T-MCV | 93-107 | DYMQSGYNARFCRGP |
| AEM01096 | Small T-MCV | 137-151 | KQKNCLTWGECFCYQ |
| AEM01096 | Small T-MCV | 97-111 | SGYNARFCRGPGCML |
| AEM01098 | VP1-MCV | 218-232 | AKLDKDGNYPIEVWC |
| AEM01099 | VP2-MCV | 129-143 | MAFSLDPLQWENSLL |

TABLE 15
Peptides for WUV

| acc | | aa | |
|---|---|---|---|
| number | gene | position | Sequence |
| ACB12038 | Large T-WUV | 17-31 | LGLDMTCWGNLPLMR |
| | Large T-WUV | 21-35 | MTCWGNLPLMRTKYL |
| | Large T-WUV | 85-99 | IPTYGTPDWDYWWSQ |
| | Large T-WUV | 89-103 | GTPDWDYWWSQFNSY |
| | Large T-WUV | 217-231 | PFRHRVSAVNNFCKG |
| | Large T-WUV | 429-443 | IVENVPKKRYWVFKG |
| | Large T-WUV | 544-558 | TMNEYLVPATLAPRF |
| | Large T-WUV | 548-562 | YLVPATLAPRFHKTV |
| | Large T-WUV | 596-610 | LIWCRPVSDFHPCIQ |
| ACB12037 | Small T-WUV | 81-95 | SSSQVECTELCCNFP |
| | Small T-WUV | 89-103 | ELCCNFPPRKYRLVG |
| | Small T-WUV | 129-143 | CNCFYCFLDKRHKQK |
| | Small T-WUV | 133-147 | YCFLDKRHKQKYKIF |
| | Small T-WUV | 141-155 | KQKYKIFRKPPMWIE |
| | Small T-WUV | 149-163 | KPPMWIECYCYRCYR |
| | Small T-WUV | 153-167 | WIECYCYRCYREWFG |
| ACB12036 | VP1-else | 103-117 | PDIPNQVSECDMLIW |
| | (WUV and KIV) | | |
| | VP1-else (WUV, | 211-225 | WVADPSRNDNCRYFG |
| | and others) | | |
| ACB12034 | VP2-WUV | 152-166 | YNLETGIPGVPDWVF |

TABLE 16
Peptides forHPyV6

| acc | | as | peptide | peptide |
|---|---|---|---|---|
| number | gene | position | sequence | sequnce aligned |
| YP_ | VP1- | 77-91 | YTLAVVNL | |
| 003848918 | HPyV6 | | PEIPEAL | |
| | VP1- | 295-309 | TVDMMFRQ | TVDMMFRQFLQPQKP |
| | HPyV6 | | FLQPQKP | |
| | VP1- | 299-313 | MFRQFLQP | -----------QVQG |
| | HPyV6 | | QKPQVQG | |
| | VP1- | 363-377 | AAGAANLF | AAGAANLFGPPVEKQ |
| | HPyV6 | | GPPVEKQ | |
| | VP1- | 367-381 | ANLFGPPV | -----------TSKE |
| | HPyV6 | | EKQTSKE | |
| | VP1- | 373-387 | PVEKQTSK | ---------PSKGEL |
| | HPyV6 | | EPSKGEL | |

TABLE 17
Peptides for HPyV7

| | peptide | | aa | |
|---|---|---|---|---|
| acc number | number | | position | ID |
| YP_003848923 | 2404 | VP1- | 51-65 | ATTGNFQS |
| | | HPyV7 | | RGLPYPM |
| | 2324 | VP1- | 51-65 | ATTGNFQS |
| | | HPyV7 | | RGLPYTM |

TABLE 18
Peptides for SV40

| ACC NUMBER | peptide number | gene | aa position | peptide sequence |
|---|---|---|---|---|
| YP_003708382 | 3661 | largeT-SV40 | 133-147 | EDPKDFPSELLSFLS |
| | 3722 | largeT-SV40 | 408-422 | FLKCMVYNIPKKRYW |
| | 3784 | largeT-SV40 | 683-697 | HNQPYHICRGFTCFK |
| | 3660 | largeT-SV40 | 129-143 | KRKVEDPKDFPSELL |
| | 3637 | largeT-SV40 | 17-31 | LGLERSAWGNIPLMR |
| | 3723 | largeT-SV40 | 412-426 | MVYNIPKKRYWLFKG |
| | 3653 | largeT-SV40 | 84-98 | PTYGTDEWEQWWNAF |
| | 3638 | largeT-SV40 | 21-35 | RSAWGNIPLMRKAYL |
| | 3783 | largeT-SV40 | 679-693 | SVHDHNQPYHICRGF |
| YP_003708383 | 4126 | smallT-SV40 | 114-128 | LLCLLRMKHENRKLY |
| YP_003708381 | 1900 | VP1-SV40 | 1-15 | MKMAPAKRKGSCPGA |
| YP_003708379 | 2755 | VP2-SV40 | 123-137 | LYRPDDYYDILFPGV |
| | 2754 | VP2-SV40 | 119-133 | MAVDLYRPDDYYDIL |

TABLE 19
The sequences of the 635
peptides mentioned in Table 6

| | |
|---|---|
| 0_05 | PLSYSRSSEEAFLEA |
| 1_00 | APKKPKEPVQVPKLL |
| 1_00 | ARFFRLHFRQRRVKN |
| 1_00 | AVGGEPLELQGVLAN |
| 1_00 | AVTVQTEVIGITSML |
| 1_00 | DKNKAYPVECWVPDP |
| 1_00 | DMKVWELYRMETELL |
| 1_00 | DMLPCYSVARIPLPN |
| 1_00 | DRKMLPCYSTARIPL |
| 1_00 | EETPDADTTVCYSLA |
| 1_00 | ELLVVPLVNALGNTN |
| 1_00 | FFAVGGEPLELQGVL |
| 1_00 | FFRLHFRQRRVKNPF |
| 1_00 | FLNPQMGNPDEHQKG |
| 1_00 | FLTPEMGDPDEHLRG |
| 1_00 | GGIEVLGVKTGVDSF |
| 1_00 | GGVEVLAAVPLSEET |
| 1_00 | KAYLDKNNAYPVECW |
| 1_00 | KRKGSCPGAAPKKPK |
| 1_00 | LDKDNAYPVECWVPD |
| 1_00 | LDKNNAYPVECWIPD |
| 1_00 | LDKNNAYPVECWVPD |
| 1_00 | LELQGVLANYRTKYP |
| 1_00 | LMNYRSKYPDGTITP |
| 1_00 | LPATVTLQATGPILN |
| 1_00 | LPGDPDMIRYIDKQG |
| 1_00 | LPGDPDMIRYIDRQG |
| 1_00 | LPGDPDMMRYVDKYG |
| 1_00 | LSDLINRRTQRVDGQ |
| 1_00 | MESQVEEVRVFDGTE |
| 1_00 | MQGVLMNYRSKYPDG |
| 1_00 | MSCTPCRPQKRLTRP |
| 1_00 | NQVSECDMLIWELYR |
| 1_00 | PDMIRYIDKQGQLQT |
| 1_00 | PDMMRYVDKYGQLQT |
| 1_00 | PLEMQGVLMNYRTKY |
| 1_00 | PYPISFLLSDLINRR |
| 1_00 | QLPRTVTLQSQTPLL |
| 1_00 | QVAPPDIPNQVSECD |
| 1_00 | RMETELLVVPLVNAL |
| 1_00 | RYFKIRLRKRSVKNP |
| 1_00 | SPERKMLPCYSTARI |
| 1_00 | TFESDSPNRDMLPCY |
| 1_00 | TLHVYNSNTPKAKVT |
| 1_00 | TSGTQQWKGLPRYFK |
| 1_00 | VECFLTPEMGDPDEH |
| 1_00 | VMNTEHKAYLDKNKA |
| 1_00 | VPLVNALGNTNGVVH |
| 1_00 | VQSQVMNTEHKAYLD |
| 1_00 | VSECDMKVWELYRME |
| 1_00 | WAPDPSRNDNCRYFG |
| 1_00 | WELYRMETELLVVPL |
| 1_00 | WVADPSRNDNCRYFG |
| 1_00 | YFGRMVGGAATPPVV |
| 1_00 | YFGTLTGGENVPPVL |
| 1_00 | YNSNTPKAKVTSERY |
| 1_00 | YSTARIPLPNLNEDL |
| 1_00 | YSVARIPLPNLNEDL |
| 1_01 | CGNLLMREAVTVKTE |
| 1_01 | DFSSDSPERKLLPCY |
| 1_01 | EHGGGKPIQGSNFHR |
| 1_01 | EKKMLPCYSTARIPL |
| 1_01 | EMGDSDENLRGFSLK |
| 1_01 | ENLRGFSLKLSAEYD |
| 1_01 | EVECFLNPEMGDSDE |
| 1_01 | FLNPEMGDSDENLRG |
| 1_01 | IPLPNLYEDLTCGNL |
| 1_01 | ITEVECFPNPEMGDP |
| 1_01 | KLSAKNDFSSDSPDR |
| 1_01 | KMLPCCSTARIPLPN |
| 1_01 | KMLPCYGTARIPLPN |
| 1_01 | KMLPCYSTTRIPLPN |
| 1_01 | KMLPCYSTVRIPLPN |
| 1_01 | KPEEPVQVPKLLIKG |
| 1_01 | LARYFKTRLRKRSVK |
| 1_01 | LARYFRIRLRKRSVK |
| 1_01 | LMREAVTVKTEVMGI |
| 1_01 | LPCYSTARIPLPNLY |
| 1_01 | LTCGNLLMWEAVTLQ |
| 1_01 | MLPCYSAARIPLPNL |
| 1_01 | MWEAATVKTEVIGIT |
| 1_01 | MWEAVQVQTEVIGIT |
| 1_01 | NLLMWEAVTVQTEVT |
| 1_01 | PLEMQGVLLNYRTKY |
| 1_01 | PLEMQGVLMNYWTKY |
| 1_01 | PNLNEDLTCENLLMW |
| 1_01 | PNLNEDLTCGNLLMR |
| 1_01 | PNLNEDLTCGNLLVW |
| 1_01 | PNLNEDLTRGNLLMW |
| 1_01 | PQRKMLPCYSTARIP |
| 1_01 | PYPISFSLSDLINRR |
| 1_01 | RIPLPNLNEDLTCEN |
| 1_01 | SFLLSDLITRRTQRV |
| 1_01 | SPERKMLPCYGTARI |
| 1_01 | TKYPHGTITPKNPTV |
| 1_01 | VSAADICGLFINSSG |
| 1_01 | YSAARIPLPNLNEDL |
| 1_01 | YSLKLTAENAFDSDS |
| 1_02 | ALELQGVVCNYRTKY |
| 1_02 | ALELQGVVFNYGTKY |
| 1_02 | ARIPLPILNEDLTCG |
| 1_02 | CGNIPMWEAVTLKTE |
| 1_02 | CWVPDPTRNENPRYF |
| 1_02 | DEFGVGLLCKGDNLY |
| 1_02 | DKTKAYPVECWVPDP |
| 1_02 | DMMRYVDRYGQLQTQ |
| 1_02 | DPDMMRYVDRYGQLQ |
| 1_02 | DPDVMRYVDRYGQLQ |
| 1_02 | DSIAEVECFLTPEMG |
| 1_02 | DTLPCYSVARIPLPN |
| 1_02 | DVLPCYSVARIPLPN |
| 1 02 | EDLTCGNIPMWEAVT |
| 1_02 | EELPEDPDMMRYVDR |
| 1_02 | EELPGDPDMIRYVDR |
| 1_02 | EELPGDPDVMRYVDR |
| 1_02 | EEVRVFEGTEGLPGD |
| 1_02 | EHKAYLDRNKAYPVE |
| 1_02 | FFLTDLINRRTPRVD |
| 1_02 | FGVGPLCKGANLYLS |
| 1_02 | FLLADLINRRTPRVD |
| 1_02 | FNYGTKYPDGTIFPK |
| 1_02 | FNYRTKYPDGTIYPK |
| 1_02 | FNYRTMYPDGTIFPK |
| 1_02 | FNYRTRYPDGTIFPK |
| 1_02 | FNYRTTYPDGPIFPK |
| 1_02 | FNYRTTYPDGTIFPK |
| 1_02 | FNYRTTYPHGTIFPK |
| 1 02 | FPLTDLINRRTPRVD |
| 1_02 | FRYFKVQLRKRRVKN |
| 1_02 | FTKRSGSQQWRGLSR |
| 1_02 | GDNLYLSAADVCGMF |
| 1_02 | GDNLYLSAVDVCDMF |
| 1_02 | GDNLYLSAVDVCGLF |
| 1_02 | GDNLYLSAVDVRGMF |
| 1_02 | GDNLYLSAVDVYGMF |
| 1_02 | GDPDMIRYVDRYGQL |
| 1_02 | GDPDMMRYVDRYGQL |
| 1_02 | GDPDMMRYVDSCRQK |
| 1_02 | GMFTNKSGSQQWRGL |
| 1_02 | GMFTNRCGSQQWRGL |
| 1_02 | GMFTNRSCSQQWRGL |
| 1_02 | IRYVDRYGQLQTKML |
| 1_02 | KNATVQSQVMNTDHK |
| 1 02 | KVELRKRRVKNPYPI |
| 1_02 | KVQLRKRKVKNPYPI |
| 1_02 | KVQLRKRRVKDPYPI |
| 1_02 | LDEFGGGPLCKGDNL |
| 1_02 | LDKNKAYPVECWGPD |
| 1_02 | LDKNKAYPVECWVPN |
| 1_02 | LINIRTPRVDGQPMY |
| 1_02 | LINRRTPGVDGQPMY |
| 1_02 | LINRRTPRVNGQPMY |
| 1_02 | LLDEFGVGPLCKGVN |
| 1_02 | LLTDLINRRTPKVDG |
| 1_02 | LLTDLINRRTPRIDG |
| 1_02 | LPGDPDMMRYVDRYG |
| 1_02 | LPILNEDLTCGNILM |
| 1_02 | MGDPDEHLRGFSKLI |
| 1_02 | MGDPNEHLRGFSKSI |
| 1_02 | MKMAPTKRKGERKDP |
| 1_02 | MMRYVDRYGQLQTKT |
| 1_02 | MMRYVDSCRQKCCNQ |
| 1_02 | MRYVDRYGQLQTQML |
| 1_02 | MRYVDRYGQSQTMML |
| 1_02 | NRSGFQQWRGLSRYF |
| 1 02 | NRSGPQQWRGLSRYF |
| 1_02 | NVPPVLHITNTASTV |
| 1_02 | NVPPVLHITNTATTA |
| 1_02 | PDMMRYVDRYGQSQT |
| 1_02 | PGDPDMMRYVDRYGQ |
| 1_02 | PNLNEDLTCGNIPMW |
| 1_02 | QPMYGMDAQVKEVRV |
| 1_02 | QSQVMNPEPKGYLDK |
| 1_02 | RKGRVKNPYPISFLL |
| 1_02 | RKRKVKNPYPISFLL |
| 1_02 | RKRRIKNPYPISFLL |
| 1_02 | RKRRVKDPYPISFLL |
| 1_02 | SKDMLPRFSVARIPL |
| 1_02 | SRYFKVELRKRRVKN |
| 1_02 | SRYFKVQLRKRKVKN |
| 1_02 | SRYFKVQLRKRRVKD |
| 1 02 | SRYFKVQPRKRRVKN |
| 1_02 | TEELPGDPDMITYVD |
| 1_02 | TIFPKNATVQSQVVN |
| 1_02 | TTGKLDEFGVGPLCK |
| 1_02 | TTVLLDDFGVGPLCK |
| 1 02 | TTVLLDEFGAGPLCK |
| 1_02 | TTVLLDEFGGGPLCK |
| 1_02 | TTVLLDEFGVRPLCK |
| 1_02 | TTVLLDELGVGPLCK |
| 1_02 | TTVLLDEYGVGPLCK |
| 1_02 | VARIPLPNINEDLTC |
| 1_02 | VARVPLPNLNEDLTC |
| 1_02 | VDSCRQKCCNQKPLL |
| 1_02 | VECFLTPEMGDPDGH |
| 1_02 | VFNYRTKYPDGPIFP |
| 1_02 | VGGEALELQGGAFNY |
| 1_02 | VGGEALELQGVAFNY |
| 1_02 | VGGEALELQGVVCNY |
| 1_02 | VGGEALELQGVVFNY |
| 1_02 | VKNPYPISFPLTDLI |
| 1_02 | VMNTEHKAYLDKNKV |
| 1_02 | VMNTEHKAYLDRNKA |
| 1_02 | VVNTEHKAYLDKNKA |
| 1_02 | WRGLSRYFKVQPRKR |
| 1_02 | WRGLSRYFRVQLRKR |
| 1_02 | YLDENKAYPVECWVP |
| 1_02 | YLDKNKVYPVECWVP |
| 1 02 | YLDRNKAYPVECWVP |
| 1_02 | YLSAVDVCGMFTDRS |
| 1_02 | YLYKNKAYPVECWVP |
| 1_02 | YPISFLLADLINRRT |
| 1_02 | YPISFPLTDLINRRT |
| 1_02 | YPITFLLTDLINRRT |
| 1_03 | KVTSERYSVEWAPDP |
| 1_03 | LWLQGRLYITCADML |
| 1_03 | MSCTACRPQKRLTRP |
| 1_03 | QLPRTVTLQSQAPLL |
| 1_03 | RMETELLVVPLVNAG |
| 1_03 | VVRGAATPPDVSYGN |
| 1_03 | YSISSAIHDKESGSI |
| 1_04 | AKLDKDGNYPIEVWC |
| 1_04 | CDTLQMWEAISVKTE |
| 1_04 | EPLPGDPDIVRFLDK |
| 1_04 | EVRIYEGSEPLPGDP |
| 1_04 | GAGIPVSGVNYHMFA |
| 1_04 | GKAPLKGPQKASQKE |
| 1_04 | GKAPLKGPQQASQKE |
| 1_04 | KASSTCKTPKRQCIP |
| 1_04 | KRWVKNPYPVVNLIN |
| 1_04 | LDENGVGPLCKGDGL |
| 1_04 | LDLQGLVLDYQTQYP |
| 1_04 | LRKRWVKNPYPVVNL |
| 1_04 | MFAIGEEPLDLQGLV |
| 1_04 | MFAIGGEPLDLQGLV |
| 1_04 | NEDITCDTLQMWEAI |
| 1_04 | NKDGNYPIEVWCPDP |
| 1_04 | PGDPDIVRFLDKFGQ |
| 1_04 | RVSLPMLNEDITCDT |
| 1_04 | SLINVHYWDMKRVHD |
| 1_04 | SPDLPTTSNWYTYTY |
| 1_04 | TTVLLDENGVGPLCK |
| 1_04 | VGISSLINVHYWVMK |
| 1_04 | VHDYGAGIPVSGVNY |
| 1_04 | VHYWVMKRVHDYGAG |
| 1_04 | YEGSEPLPGDPDIVR |
| 1_04 | APKKPKEPVQVPKLV |
| 1_05 | AVVGEPLELQGVLAN |
| 1_05 | KMAPAKRKGSCPGAA |
| 1_05 | MKMAPAKRKGSCPGA |
| 1_05 | MKMAPTKRKGSCPGA |
| 1_05 | TTVLLDEQGAGPLCK |
| 1_06 | GSHMGGVDVLAAVPL |
| 1_06 | KGGVDVLSAVPLSEE |
| 1_06 | MACTAKPACTPKPGR |
| 1_06 | NQVSECDMIIWELYR |
| 1_06 | PDIPNQVSECDMIIW |
| 1_07 | AITQIEAYLNPRMGN |
| 1_07 | ATTPPVMQFTNSVTT |
| 1_07 | DIVGIHTNYSESQNW |
| 1_07 | EGLPGDPDLDRYVDK |
| 1_07 | FTGGATTPPVMQFTN |
| 1_07 | IEAYLNPRMGNNNPT |
| 1_07 | KTCPTPAPVPKLLVK |
| 1_07 | LNPRMGNNNPTDELY |
| 1_07 | MWEAVSVKTEVMGIS |
| 1_07 | SDNPNATTLPTYSVA |
| 1_07 | SGLMPQIQGQPMEGT |
| 1_07 | VQGTTLHMFSVGGEP |
| 1_07 | VSVKTEVMGISSLVN |
| 1_07 | YPTDMVTIKNMKPVN |
| 1_07 | YSESQNWRGLPRYFN |
| 1_08 | ENTRYYGSYTGGQST |
| 1_08 | GEPLELQFLTGNYRT |
| 1_08 | GLPRYFNILLRKRTV |
| 1_08 | GTEGLPGDPDMVRYI |
| 1_08 | GVSSLVNVHMATKRM |
| 1_08 | HMATKRMYDDKGIGF |
| 1_08 | IELYLNTRMGQNDES |
| 1_08 | IGFPVEGMNFHMFAV |
| 1_08 | KDGDMQYRGLPRYFN |
| 1_08 | KFGQDKTRPPFPARL |
| 1_08 | KQKLIKDGAFPVECW |
| 1_08 | LPGDPDMVRYIDKFG |
| 1_08 | LSTQVEEVRVYDGTE |
| 1_08 | LVNVHMATKRMYDDK |
| 1_08 | PDMVRYIDKFGQDKT |
| 1_08 | PVLQFTNTVITVLLD |
| 1_08 | QSTPPVLQFTNTVIT |
| 1_08 | RTVRNPYPVSSLLNN |
| 1_08 | RYIDKFGQDKTRPPF |
| 1_08 | TEVVGVSSLVNVHMA |
| 1_08 | TKDGAFPVECWCPDP |
| 1_08 | TQGLNPHYKQKLTKD |
| 1_08 | VEGMNFHMFAVGGEP |
| 1_08 | VSVKTEVVGVSSLVN |
| 1_08 | YRTDYSANDKLVVPP |
| 1_08 | YYGSYTGGQSTPPVL |
| 1_09 | AAGAANLFGPPVEKQ |
| 1_09 | ANLFGPPVEKQTSKE |
| 1_09 | CGGSPLDVIGINPDP |
| 1_09 | EDTIYKVEAILLPNF |
| 1_09 | ETELIFTPQVGSAGY |
| 1_09 | FLQPQKPQVQGTQPN |
| 1_09 | GNPTLSDAYSQQRSV |
| 1_09 | MFRQFLQPQKPQVQG |
| 1_09 | MLGMVGYAGNPTLSD |
| 1_09 | NQSTTPLVDENGVGI |
| 1_09 | PVEKQTSKEPSKGEL |
| 1_09 | QKPQVQGTQPNAVQE |
| 1_09 | TAVYQSRGAPYTFTD |
| 1_09 | TRKQVTAANFPIEIW |
| 1_09 | TVDMMFRQFLQPQKP |
| 1_09 | VTAANFPIEIWSADP |
| 1_09 | YKVEAILLPNFASGS |
| 1_09 | YTLAVVNLPEIPEAL |
| 1_10 | AKISVAPKKNTDKKE |
| 1_10 | APTSKFLLQNGELIY |
| 1_10 | ATTGNFQSRGLPYPM |
| 1_10 | ATTGNFQSRGLPYTM |
| 1_10 | DAMCEDTMIVWEAYR |
| 1_10 | EDTMIVWEAYRLETE |
| 1_10 | FFRVHCRQRRIKHPY |
| 1_10 | GPLDVIGINPDPERL |
| 1_10 | ISVAPKKNTDNKKEL |
| 1_10 | RKQVNAANFPVELWV |
| 1_10 | WACGGGPLDVIGINP |
| 2_01 | ANQRTAPQWMLPLLL |
| 2_01 | EYYSDLSPIRPSMVR |
| 2_01 | MALELFNPDEYYDIL |
| 2_01 | WHVIRDDIPAITSQE |
| 2_01 | AFVNNIHYLDPRHWG |
| 2_02 | ANQRSAPQWMLPLLL |
| 2_02 | APGGANQRSAPQWML |
| 2_02 | EDYYDILFPGVNAFV |
| 2_02 | KVSTVGLFQQPAMAL |
| 2_02 | MALQLFNPEDYYDIL |
| 2_02 | NLVRDDLPSLTSQEI |
| 2_02 | PGVNAFVNNIHYLDP |
| 2_02 | YLDPRHWGPSLFSTI |
| 2_02 | YYSRLSPVRPSMVRQ |
| 2_03 | FNALSEGVHRLGQWI |
| 2_03 | GLAALGGITEGAALL |
| 2_03 | KRKQDELHPVSPTKK |
| 2_03 | LPELPSLQDVFNRIA |
| 2_03 | LVASYLPELPSLQDV |
| 2_03 | MALVPIPEYQLETGI |
| 2_03 | PIPEYQLETGIPGIP |
| 2_03 | PSLQDVFNRIAFGIW |
| 2_03 | PVNAIATQVRSLATT |
| 2_03 | TGGTPHYATPDWILY |
| 2_03 | VHKPIHAPYSGMALV |
| 2_03 | VLSDEIQRLLRDLEY |
| 2_03 | YQLETGIPGIPDWLF |
| 2_04 | HIGGTLQQQTPDWLL |
| 2_04 | LDPLQWENSLLHSVG |
| 2_04 | MAFSLDPLQWENSLL |
| 2_04 | QWENSLLHSVGQDIF |
| 2_04 | RHALMAFSLDPLQWE |
| 2_04 | TLQQQTPDWLLPLVL |
| 2_05 | ADSIQQVTERWEAQS |
| 2_05 | APQWMLPLLLGLYGS |
| 2_05 | DDYYDILFPGVQTFV |
| 2_05 | KAYEDGPNKKKRKLS |
| 2_05 | MAVDLYRPDDYYDIL |
| 2_05 | PGVQTFVHSVQYLDP |
| 2_05 | QDYYSTLSPIRPTMV |
| 2_05 | SVQYLDPRHWGPTLF |
| 2_05 | TTWTVINAPVNWYNS |
| 2_06 | DVFNRIAYGIWTSYY |
| 2_06 | ELQRLLGDLEYGFRT |
| 2_06 | FIASHLPELPSLQDV |
| 2_06 | GGIYTALAADRPGDL |
| 2_06 | GIWTSYYNTGRTVVN |
| 2_06 | GLAALGGLTESAALL |
| 2_06 | LLGDLEYGFRTALAT |
| 2_06 | MALAPIPEYNLETGI |
| 2_06 | PDWILYVLEELNSDI |
| 2_06 | PIPEYNLETGIPGVP |
| 2_06 | PSLQDVFNRIAYGIW |
| 2_06 | RERELLQIAAGQPVD |
| 2_06 | RIAYGIWTSYYNTGR |
| 2_06 | VVNRAVSEELQRLLG |
| 2_06 | YNLETGIPGVPDWVF |
| 2_12 | ASLATVEGITTTSEA |
| 2_12 | DDLPSLTSREIQRRT |
| 2_12 | DYYSNLSPIRPSMVR |
| 2_12 | IAGFAALIQTVTGVS |
| 2_12 | LLGLYGTVTPALAAY |
| 2_12 | NLVRDDLPALTSQEI |
| 2_12 | NLVRDDLPSLTSREI |
| 2_12 | QQPVMALQLFNPEDY |
| 3_04 | TVGVRLSREQVSLVN |
| 4_01 | AIDQYMVVFEDVKGT |
| 4_01 | CLLPKMDSVIFDFLH |
| 4_01 | DFATDIQSRIVEWKE |
| 4_01 | DIQSRIVEWKERLDS |
| 4_01 | EELHLCKGFQCFKRP |
| 4_01 | ELGVAIDQYMVVFED |
| 4_01 | ESMELMDLLGLERAA |
| 4_01 | EYLLYSALTRDPYHT |
| 4_01 | FFLTPHRHRVSAINN |
| 4_01 | FLHCIVFNVPKRRYW |
| 4_01 | GGDEDKMKRMNTLYK |
| 4_01 | HGINNLDSLRDYLDG |
| 4_01 | ISMYTFSRMKYNICM |
| 4_01 | KRVDTLHMTREEMLT |
| 4_01 | KYSVTFISRHMCAGH |
| 4_01 | LCKGFQCFKRPKTPP |
| 4_01 | LDSEISMYTFSRMKY |
| 4_01 | LGLERAAWGNLPLMR |
| 4_01 | LMRKAYLRKCKEFHP |
| 4_01 | LNREESMELMDLLGL |
| 4_01 | MAGVAWLHCLLPKMD |
| 4_01 | MDKVLNREESMELMD |
| 4_01 | RVSAINNFCQKLCTF |
| 4_01 | TFSRMKYNICMGKCI |
| 4_01 | VKVNLEKKHLNKRTQ |
| 4_02 | CGGKSLNVNMPLERL |
| 4_02 | EHCTYHICKGFQCFK |
| 4_02 | FLKCIVLNIPKKRYW |
| 4_02 | GNIPVMRKAYLKKCK |
| 4_02 | HFNHHEKHYYNAQIF |
| 4_02 | ISNLDCLRDYLDGSV |
| 4_02 | KGFQCFKKPKTPPPK |
| 4_02 | LLDLCGGKSLNVNMP |
| 4_02 | LMDLLGLDRSAWGNI |
| 4_02 | MKANVGMGRPILDFP |
| 4_02 | RKHQNKRTQVFPPGI |
| 4_02 | RVSAINNYCQKLCTF |
| 4_02 | SGHGISNLDCLRDYL |
| 4_02 | TKCEDVFLLMGMYLD |
| 4_02 | TMNEYSVPRTLQARF |
| 4_02 | VDSIHMTREEMLVER |
| 4_02 | VGMGRPILDFPREED |
| 4_02 | VNLERKHQNKRTQVF |
| 4_02 | VPTYGTDEWESWWNT |
| 4_02 | WESWWNTFNEKWDED |
| 4_02 | WNTFNEKWDEDLFCH |
| 4_02 | ALDQYMVVFEDVKGQ |
| 4_03 | ALEFDIDDVYYLLGS |
| 4_03 | CSQATPPKKKHAFDA |
| 4_03 | EDLFGEPKEPSLSWN |
| 4_03 | EFVSHAVFSNKCITC |
| 4_03 | EIQSNVVYWKEVLDN |
| 4_03 | ELGVALDQYMVVFED |
| 4_03 | FFLTPHKHRVSAINN |
| 4_03 | FLFCKGVNNPYGLYS |
| 4_03 | GEPKEPSLSWNQIAN |
| 4_03 | GNGVNNLDNLRDYLD |
| 4_03 | HKRVHVQNHENAVLL |
| 4_03 | IPGGLKENEFNPEDL |
| 4_03 | IPTYGTPDWDEWWSQ |
| 4_03 | KGVNNPYGLYSRMCR |
| 4_03 | LDNYIGLTEFATMQM |
| 4_03 | LKENEFNPEDLFGEP |
| 4_03 | LNINIPSEKLPFELG |
| 4_03 | LQKYQCSFISKHAFY |
| 4_03 | NKRSQIFPPGIVTMN |
| 4_03 | NNLDNLRDYLDGCVE |
| 4_03 | NPYGLYSRMCRQPFN |
| 4_03 | NVVYWKEVLDNYIGL |
| 4_03 | PGIVTMNEYCIPETV |
| 4_03 | PHKHRVSAINNFCKG |
| 4_03 | QCSFISKHAFYNTVL |
| 4_03 | TMNEYCIPETVAVRF |
| 4_03 | VHDLNEEEDNIWQSS |
| 4_03 | YKKLLQKYQCSFISK |
| 4_03 | YMASIAWYTGLNKKI |
| 4_04 | AIELYDKIEKFKVDF |
| 4_04 | AIYTTSDKAIELYDK |
| 4_04 | AVSLEKKHVNKKHQI |
| 4_04 | CKKFKKHLERLRDLD |
| 4_04 | CKPPYKLLQENKPLL |
| 4_04 | CLIWCLPDTTFKPCL |
| 4_04 | CLPDTTFKPCLQEEI |
| 4_04 | DLDTIDLLYYMGGVA |
| 4_04 | EKKLQKIIQLLTENI |
| 4_04 | ENIPKYRNIWFKGPI |
| 4_04 | ERLRDLDTIDLLYYM |
| 4_04 | HSQSSSSGYGSFSAS |
| 4_04 | KPFPCQKCENRSRLK |
| 4_04 | LAHYLDFAKPFPCQK |
| 4_04 | LCKLLEIAPNCYGNI |
| 4_04 | LEIAPNCYGNIPLMK |
| 4_04 | MDLVLNRKEREALCK |
| 4_04 | MPEMYNNLCKPPYKL |
| 4_04 | NKDLQPGQGINNLDN |
| 4_04 | NSSRTDGTWEDLFCD |
| 4_04 | PCLQEEIKNWKQILQ |
| 4_04 | QLLTENIPKYRNIWF |
| 4_04 | SVPRNSSRTDGTWED |
| 4_04 | TDGTWEDLFCDESLS |
| 4_04 | TPVPTDFPIDLSDYL |
| 4_04 | TSDKAIELYDKIEKF |
| 4_04 | VDFKSRHACELGCIL |
| 4_04 | YKLLQENKPLLNYEF |
| 4_04 | YRSSSFTTPKTPPPF |
| 4_05 | AWLHCLLPKMDSVVY |
| 4_05 | CLLPKMDSVVYDFLK |
| 4_05 | EDPKDFPSELLSFLS |
| 4_05 | EFAQSIQSRIVEWKE |
| 4_05 | EKMKKMNTLYKKMED |
| 4_05 | ELGVAIDQFLVVFED |
| 4_05 | EYLMYSALTRDPFSV |
| 4_05 | FGGFWDATEIPTYGT |
| 4_05 | FGSTGSADIEEWMAG |
| 4_05 | FLKCMVYNIPKKRYW |
| 4_05 | GNIPLMRKAYLKKCK |
| 4_05 | GQGINNLDNLRDYLD |
| 4_05 | HNQPYHICRGFTCFK |
| 4_05 | KEKAALLYKKIMEKY |
| 4_05 | KMNTLYKKMEDGVKY |
| 4_05 | KRKVEDPKDFPSELL |
| 4_05 | LGLERSAWGNIPLMR |
| 4_05 | LMRKAYLKKCKEFHP |
| 4_05 | MDKVLNREESLQLMD |
| 4_05 | MLTNRFNDLLDRMDI |
| 4_05 | MVYNIPKKRYWLFKG |
| 4_05 | PTYGTDEWEQWWNAF |
| 4_05 | RSAWGNIPLMRKAYL |
| 4_05 | RVSAINNYAQKLCTF |
| 4_05 | SFQAPQPSQSSQSVH |
| 4_05 | SVHDHNQPYHICRGF |
| 4_05 | TREQMLTNRENDLLD |
| 4_05 | VKYAHQPDFGGFWDA |
| 4_05 | VTEYAMETKCDDVLL |
| 4_05 | WDATEIPTYGTDEWE |
| 4_05 | YHICRGFTCFKKPPT |
| 4_06 | AAALLDLCGGKALNI |
| 4_06 | AWYLGLNGKIDELVY |
| 4_06 | CVSTVHQLNEEEDEV |
| 4_06 | EDLLARRFEKILDKM |
| 4_06 | EEKMKKLNSLYLKLQ |
| 4_06 | EFVSQAVFSNRTLTA |
| 4_06 | EKILDKMDKTIKGEQ |
| 4_06 | ELGVAIDQFTVVFED |
| 4_06 | ESLDKTPELMVKRVL |
| 4_06 | FILTPFRHRVSAVNN |
| 4_06 | GEFKDQLNWKALSEF |
| 4_06 | GEQDVLLYMAGVAWY |
| 4_06 | GLNGKIDELVYRYLK |
| 4_06 | GNGMSNLDNLRDYLD |
| 4_06 | GNLPLMRTKYLSKCK |
| 4_06 | GTPDWDYWWSQFNSY |
| 4_06 | IPTYGTPDWDYWWSQ |
| 4_06 | IVENVPKKRYWVFKG |
| 4_06 | KCNFASRHSYYNTAL |
| 4_06 | KDNATDASLSFPKEL |
| 4_06 | LDKYIGLTEFADMQM |
| 4_06 | LEKKHLNKRSQIFPP |
| 4_06 | LGLDMTCWGNLPLMR |
| 4_06 | LIWCRPVSDFHPCIQ |
| 4_06 | LKENDFKAEDLYGEF |
| 4_06 | MTCWGNLPLMRTKYL |
| 4_06 | NAYGLYSRMTRDPFT |
| 4_06 | PFRHRVSAVNNFCKG |
| 4_06 | PGIVTMNEYLVPATL |
| 4_06 | PKKKKDNATDASLSF |
| 4_06 | SSSQIPTYGTPDWDY |
| 4_06 | TMNEYLVPATLAPRF |
| 4_06 | VIHTTKEKAETLYKK |
| 4_06 | VKVNLEKKHLNKRSQ |
| 4_06 | YLVPATLAPRFHKTV |
| 4_12 | CGGKSLNVNMPLEKL |
| 4_12 | DFAQDIQSRIVEWKE |
| 4_12 | DSGHGSSTESQSQCC |
| 4_12 | EYLLYSALTRDPYYI |
| 4_12 | EYLLYSALTREPYHT |
| 4_12 | GSVRVNLERKHQNKR |
| 4_12 | GVNKEYLLYSALTRE |
| 4_12 | HMTREEMLVQRFNFL |
| 4_12 | KRVDSLHMTREEMLT |
| 4_12 | LGLDRSAWGNIPIMR |
| 4_12 | MLTDRFNHILDKMDL |
| 4_12 | SLHMTREEMLTDRFN |
| 4_12 | VDSIHMTREEMLVQR |
| 4_12 | YLRKSLSSSEYLLEK |
| 5_01 | CADFPLCPDTLYCKE |
| 5_01 | LRHLNRKFLRKEPLV |
| 5_01 | PDFGTWSSSEVCADF |
| 5_01 | SSEVCADFPLCPDTL |
| 5_02 | CDFPPNSDTLYCKEW |
| 5_02 | FGTWNSSEVGCDFPP |
| 5_02 | IDCYCFDCFRQWFGC |
| 5_02 | LMCMLKLRHRNRKFL |
| 5_02 | NCATNPSVHCPCLMC |
| 5_03 | CIHGYNHECQCIHCI |
| 5_03 | CYREWFFFPISMQTF |
| 5_03 | FWKVIIFNTEIRAVQ |
| 5_03 | HCILSKYHKEKYKIY |
| 5_03 | KPPVWIECYCYKCYR |
| 5_03 | QSSQVYCKDLCCNKF |
| 5_03 | WIECYCYKCYREWFF |
| 5_03 | YCYKCYREWFFFPIS |
| 5_03 | YIMKQWDVCIHGYNH |
| 5_03 | YYEAYIMKQWDVCIH |
| 5_04 | DYMQSGYNARFCRGP |
| 5_04 | FGFPPTWESFDWWQK |
| 5_04 | FSMFDEVSTKFPWEE |
| 5_04 | GTLKDYMQSGYNARF |
| 5_04 | KQKNCLTWGECFCYQ |
| 5_04 | RGPGCMLKQLRDSKC |
| 5_04 | SGYNARFCRGPGCML |
| 5_04 | WQKTLEETDYCLLHL |
| 5_05 | HQPDFGGFWDATEVF |
| 5_05 | LLCLLRMKHENRKLY |
| 5_05 | LRMKHENRKLYRKDP |
| 5_05 | PGVDAIYCKQWPECA |
| 5_06 | CNCFYCFLDKRHKQK |
| 5_06 | ELCCNFPPRKYRLVG |
| 5_06 | FYYLCNCFYCFLDKR |
| 5_06 | KIFRKPPMWIECYCY |
| 5_06 | KPPMWIECYCYRCYR |
| 5_06 | KQKYKIFRKPPMWIE |
| 5_06 | SSSQVECTELCCNFP |
| 5_06 | YCFLDKRHKQKYKIF |
| 5_06 | YCYRCYREWFGFEIS |
| 5_12 | CADFPLCPDTLYCKD |
| 5_12 | EKMKKMNTLYKKMEQ |
| 5_12 | FGTWNSSEVCADFPL |
| 5_12 | FGTWNSSEVSCDFPP |
| 5_12 | GNLSLMRKAYLRKCK |
| 5_12 | HCPCLMCMLKLRHKN |
| 5_12 | IDCYCFDCFRQWFGL |
| 5_12 | LGLERAAWGNLSLMR |
| 5_12 | LMCMLKLRHKNRKFL |
| 5_12 | PLCPDTLYCKDWPIC |
| 5_12 | RAAWGNLSLMRKAYL |
| 6_01 | LLEFCRGEDSVDGKN |
| 6_01 | QASVKVSKTWTGTKK |
| 6_05 | KVRRSWTESKKTAQR |
| 6_12 | LLEFCRGKDSVDGKN |
| 6_12 | MVLRQLSRQASVKIG |
| 6_12 | QASVKIGKTWTGTKK |

TABLE 20
Gene ID for polyomavirus peptides

| | Gene ID for polyomaviruses | | | | | | | |
|---|---|---|---|---|---|---|---|---|
| | BKV | JCV | KIV | MCV | SV40 | WUV | HPyV6 | HPyV7 |
| VP1 | _1_01 | _1_02 | _1_03 | _1_04 | _1_05 | _1_06 | _1_09 | _1_10 |
| VP2 | _2_01 | _2_02 | _2_03 | _2_04 | _2_05 | _2_06 | _2_09 | _2_10 |
| large T | _4_01 | _4_02 | _4_03 | _4_04 | _4_05 | _4_06 | _4_09 | _4_10 |
| small T | _5_01 | _5_02 | _5_03 | _5_04 | _5_05 | _5_06 | _5_09 | _5_10 |

1. Human polyoma virus peptide sequences possessing an activity towards human antibodies in human samples.

2. Human polyoma virus peptide sequences according to claim 1 having any of the sequences as indicated in Table 11 to Table 18.

3. Human polyoma virus peptide sequences according to claim 1 having any of the sequences as indicated in Table 9.

4. Use of human polyoma virus peptide sequences according to claim 2 or 3 for immune diagnostic purposes.

5. Use of human polyoma virus peptide sequences according to claim 3 for B-cell epitope studies.

6. The use of human polyoma virus peptide sequences according to claim 3 for B-cell stimulation and B-cell functionality studies.

7. A device comprising a human polyoma virus peptide sequence according to claim 2 or 3.

8. Use of human polyoma viral small T antigen for immune response diagnostic purposes.
